SEQUENCE LISTING (55 sequences)

SEQ ID NO: 1 - protein, 143 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 2 - protein, 22 aa, Homo sapiens
MQHRGFLLLTLLALLALTSAVA

SEQ ID NO: 3 - protein, 121 aa, Homo sapiens
KKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 4 - DNA, 786 bp, Homo sapiens
cgggcgaagcagcgcgggcagcgagatgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactagacgccaagcctggatgccaaggagcccctggtgtcacatggggcctggccacgccctccctctcccaggcccgagatgtgacccaccagtgccttctgtctgctcgttagctttaatcaatcatgccctgccttgtccctctcactccccagccccacccctaagtgcccaaagtggggagggacaagggattctgggaagcttgagcctcccccaaagcaatgtgagtcccagagcccgcttttgttcttccccacaattccattactaagaaacacatcaaataaactgactttttccccccaataaaagctcttcttttttaatat

SEQ ID NO: 5 - DNA, 21 bp, Homo sapiens
aagaaagataaggtgaagaag

SEQ ID NO: 6 - RNA, 21 nt, Homo sapiens
gaaagauaaggugaagaaguu

SEQ ID NO: 7 - RNA, 21 nt, Homo sapiens
uucuuucuauuccacuucuuc

SEQ ID NO: 8 - DNA, 21 bp, Homo sapiens
aactggaagaaggagtttgga

SEQ ID NO: 9 - RNA, 21 nt, Homo sapiens
cuggaagaaggaguuuggauu

SEQ ID NO: 10 - RNA, 21 nt, Homo sapiens
uugaccuucuuccucaaaccu

SEQ ID NO: 11 - DNA, 21 bp, Homo sapiens
aagtacaagtttgagaactgg

SEQ ID NO: 12 - RNA, 21 nt, Homo sapiens
guacaaguuugagaacugguu

SEQ ID NO: 13 - RNA, 21 nt, Homo sapiens
uucauguucaaacucuugacc

SEQ ID NO: 14 - DNA, 21 bp, Homo sapiens
aagaccaaagcaaaggccaaa

SEQ ID NO: 15 - RNA, 21 nt, Homo sapiens
gaccaaagcaaaggccaaauu

SEQ ID NO: 16 - RNA, 21 nt, Homo sapiens
uucugguuucguuuccgguuu

SEQ ID NO: 17 - DNA, 21 bp, Homo sapiens
aaagcaaaggccaaagccaag

SEQ ID NO: 18 - RNA, 21 nt, Homo sapiens
agcaaaggccaaagccaaguu

SEQ ID NO: 19 - RNA, 21 nt, Homo sapiens
uuucguuuccgguuucgguuc

SEQ ID NO: 20 - DNA, 21 bp, Homo sapiens
aagcaaaggccaaagccaaga

SEQ ID NO: 21 - RNA, 21 nt, Homo sapiens
gcaaaggccaaagccaagauu

SEQ ID NO: 22 - RNA, 21 nt, Homo sapiens
uucguuuccgguuucgguucu

SEQ ID NO: 23 - DNA, 21 bp, Homo sapiens
aaaggccaaagccaagaaagg

SEQ ID NO: 24 - RNA, 21 nt, Homo sapiens
aggccaaagccaagaaagguu

SEQ ID NO: 25 - RNA, 21 nt, Homo sapiens
uuuccgguuucgguucuuucc

SEQ ID NO: 26 - DNA, 21 bp, Homo sapiens
caactggaagaaggagtttgg

SEQ ID NO: 27 - RNA, 21 nt, Homo sapiens
acuggaagaaggaguuugguu

SEQ ID NO: 28 - RNA, 21 nt, Homo sapiens
uuugaccuucuuccucaaacc

SEQ ID NO: 29 - DNA, 21 bp, Homo sapiens
caagtacaagtttgagaactg

SEQ ID NO: 30 - RNA, 21 nt, Homo sapiens
aguacaaguuugagaacuguu

SEQ ID NO: 31 - RNA, 21 nt, Homo sapiens
uuucauguucaaacucuugac

SEQ ID NO: 32 - DNA, 21 bp, Homo sapiens
caagaccaaagcaaaggccaa

SEQ ID NO: 33 - RNA, 21 nt, Homo sapiens
agaccaaagcaaaggccaauu

SEQ ID NO: 34 - RNA, 21 nt, Homo sapiens
uuucugguuucguuuccgguu

SEQ ID NO: 35 - DNA, 21 bp, Homo sapiens
caaagcaaaggccaaagccaa

SEQ ID NO: 36 - RNA, 21 nt, Homo sapiens
aagcaaaggccaaagccaauu

SEQ ID NO: 37 - RNA, 21 nt, Homo sapiens
uuuucguuuccgguuucgguu

SEQ ID NO: 38 - DNA, 21 bp, Homo sapiens
caaaggccaaagccaagaaag

SEQ ID NO: 39 - RNA, 21 nt, Homo sapiens
aaggccaaagccaagaaaguu

SEQ ID NO: 40 - RNA, 21 nt, Homo sapiens
uuuuccgguuucgguucuuuc

SEQ ID NO: 41 - DNA, 21 bp, Homo sapiens
gaagaaggagtttggagccga

SEQ ID NO: 42 - RNA, 21 nt, Homo sapiens
agaaggaguuuggagccgauu

SEQ ID NO: 43 - RNA, 21 nt, Homo sapiens
uuucuuccucaaaccucggcu

SEQ ID NO: 44 - DNA, 21 bp, Homo sapiens
gagccgactgcaagtacaagt

SEQ ID NO: 45 - RNA, 21 nt, Homo sapiens
gccgacugcaaguacaaguuu

SEQ ID NO: 46 - RNA, 21 nt, Homo sapiens
uucggcugacguucauguuca

SEQ ID NO: 47 - DNA, 21 bp, Homo sapiens
gactgcaagtacaagtttgag

SEQ ID NO: 48 - RNA, 21 nt, Homo sapiens
cugcaaguacaaguuugaguu

SEQ ID NO: 49 - RNA, 21 nt, Homo sapiens
uugacguucauguucaaacuc

SEQ ID NO: 50 - DNA, 21 bp, Homo sapiens
gaccaaagcaaaggccaaagc

SEQ ID NO: 51 - RNA, 21 nt, Homo sapiens
ccaaagcaaaggccaaagcuu

SEQ ID NO: 52 - RNA, 21 nt, Homo sapiens
uugguuucguuuccgguuucg

SEQ ID NO: 53 - DNA, 17 bp, Homo sapiens
cggtgggcaagcgaagt

SEQ ID NO: 54 - DNA, 20 bp, Homo sapiens
cccctggtctaggcctgtct

SEQ ID NO: 55 - DNA, 24 bp, Homo sapiens
agagctgacaggctgcgagaggga

(19) United States
(12) Patent Application Publication
(10) Pub. No.: US 2005/0214292 A1
(43) Pub. Date: Sep. 29, 2005

(54) Compositions and methods for diagnosing and treating autoimmune disease

(75) Inventors: Margot Mary O'Toole, Newton, MA (US); Wei Liu, Sudbury, MA (US)

Correspondence Address: NIXON PEABODY, LLP, 401 9TH STREET, NW, SUITE 900, WASHINGTON, DC, 20004-2128, US

(73) Assignee: Wyeth, Madison, NJ (US)

(21) Appl. No.: 10/686,619

(22) Filed: Oct. 17, 2003

Related U.S. Application Data
(60) Provisional application No. 60/419,088, filed on Oct. 18, 2002.

(51) Int. Cl. A61K 48/00; C12Q 1/68; G01N 33/53; A61K 39/395

(52) U.S. Cl. 424/145.1; 514/44; 435/6; 435/7.1

(57) ABSTRACT

The present invention is generally directed to compositions and methods for the diagnosis, treatment, and prevention of lupus nephritis (LN), to the identification of novel therapeutic agents for LN, and to the creation of cell lines and animal models for studying the pathogenesis of the disease. The present invention is based on the discovery of transcribed polynucleotides that are either over-expressed or under-expressed in animals that develop lupus or are pre-disposed to lupus.

The present application incorporates by reference U.S. Provisional Application Ser. No. 60/419,088, filed Oct. 18, 2002 and entitled “Compositions and Methods for Diagnosing and Treating Autoimmune Disease.”

### FIELD OF THE INVENTION

The present invention relates generally to diagnosis and treatment of autoimmune diseases. The invention specifically relates to diagnosing and treating systemic lupus erythematosus (SLE) and lupus nephritis (LN) by monitoring and modulating, respectively, midkine (MDK) activity or MDK gene expression.

### BACKGROUND

Lupus nephritis (LN) is an inflammation of the kidney caused by systemic lupus erythematosus (SLE). SLE, commonly known as lupus, is an autoimmune rheumatic disease characterized by deposition in tissues of autoantibodies and immune complexes leading to tissue injury. In contrast to autoimmune diseases such as multiple sclerosis and type 1 diabetes mellitus, SLE potentially involves multiple organ systems directly, and its clinical manifestations are diverse and variable. For example, some patients may demonstrate primarily skin rash and joint pain, show spontaneous remissions, and require little medication. At the other end of the spectrum are patients who demonstrate severe and progressive kidney involvement that requires immediate medical attention.

The serological hallmark of SLE, and the primary diagnostic test available until now, is elevated serum levels of IgG antibodies to constituents of the cell nucleus, such as double-stranded DNA (dsDNA), single-stranded DNA (ss-DNA), and chromatin. Among these autoantibodies, IgG anti-dsDNA antibodies play a major role in the development of LN. LN is a serious condition in which the capillary walls of the kidney's blood purifying glomeruli become thickened by accretions on the epithelial side of glomerular basement membranes. The disease is often chronic and progressive and may lead to eventual renal failure.

SLE is predominantly a female disease with an approximate female to male ratio of 9:1. In North America, it is estimated to affect 1 in 500 females between the age of 20 to 40 years. It has been estimated that 45-75% of SLE patients eventually suffer kidney damage.

SLE shows a strong familial aggregation. While genetically determined immune abnormalities are implicated in the cause of SLE, the triggering event is suggested to include both exogenous and endogenous factors, likely mutagenic in origin. Certain environmental and pharmacologic agents, including UV light and drugs, such as procainamide and hydralazine have been shown to trigger a lupus-like illness in genetically predisposed individuals.

Genetic studies of murine SLE have identified susceptibility loci in several inbred strains which spontaneously develop LN (Reviewed in A. N. Theofilopoulus, The basis of autoimmunity: Part II. Genetic predisposition, Immunology Today 15:150-58, 1995). These studies have included genome-wide searches for evidence of linkage using backcrosses or F₂ intercrosses of lupus mice such as MRL/LPR, NZB/NZW and NZM/Aeg2410 mice. Recent success in mapping a susceptibility locus for multiple sclerosis in the 5p14-p12 region, which is syngenic to the murine locus Ea2, further supports the utility of this mouse-to-human approach. A genetic marker test for lupus has been generally described by Tsao et al. in U.S. Pat. No. 6,280,941.

MRL/MpJ-Fas^{lpr} mouse is a model for systemic lupus erythematosus-like autoimmune syndromes. The MRL/MpJ-Fas^{lpr} mice are generated by introducing a lymphoproliferation spontaneous mutation (Fas^{lpr}) within the fas gene into the MRL/MpJ mice. The fas protein is a cell surface antigen of about 35 kD that mediates apoptosis. It has a single transmembrane domain between its extracellular and cytoplasmic domains. The fas protein shows structural homology with several cell surface antigens, including the tumor necrosis factor and the low-affinity nerve growth factor receptor and is considered a member of the tumor necrosis factor receptor superfamily. The ligand for the fas protein, encoded by Fasl, is a member of the tumor necrosis factor family. Fas and its ligand are also involved in down-regulating immune reactions.

MRL/MpJ-Fas^{lpr} mice show systemic autoimmunity, massive lymphadenopathy associated with proliferation of aberrant T cells, arthritis, and LN. Onset and severity of symptoms is dependent on genetic background, with the original MRL/MpJ background being most severely affected beginning about 8 weeks of age. Females die at an average age of 17 weeks of age and males at 22 weeks. It has been demonstrated that the Fas^{lpr} mutation is required for the development of LN and the subsequent death at an early age.

MRL/MpJ mice, the ancestral strain of MRL/MpJ-Fas^{lpr}, also exhibit autoimmune disorders but the symptoms are manifested much later in life compared to those of the MRL/MpJ-Fas^{lpr} mice. Starting at about three months of age, levels of circulating immune complexes rise greatly in the MRL/MpJ-Fas^{lpr} mouse but not in the wildtype control, MRL/MpJ. Also, beginning at 3 months MRL/MpJ-Fas^{lpr} mice exhibit very severe proliferative glomerulonephritis, whereas in the MRL/MpJ controls usually only mild glomerular lesions are detected. The MRL/MpJ lymphoproliferation wild type females die at 73 weeks of age and males at 93 weeks, as in contrast to a lifespan of 17 weeks in the female and 22 weeks for males in the MRL/MpJ mouse homozygous for Fas^{lpr}. However, when the Fas^{lpr} mutation is bred into other strains (C57BL/6 for example), kidney function remains normal through life. It thus appears that the MRL/MpJ mice have inherited a predisposition to developing lupus which is accelerated in the presence of the Fas^{lpr} allele.

NZB×NZW F1 mouse is another animal model that develops an autoimmune disease resembling human SLE, with high titers of natural thymocyto-toxic autoantibody. NZB×NZW F1 hybrid B cells apparently differ from normal murine B cells in their capacity to produce IgG antibodies upon T cell-dependent antigenic stimulation. Genetic analysis of a backcross to NZW shows that one set of loci regulate serum levels of IgG antibodies to double-stranded DNA, single-stranded DNA, total histones and chromatin. These loci overlap with a second set of loci that control autoantibodies to the viral glycoprotein gp70. The second set of loci are most strongly linked with renal disease. A locus on distal chromosome 4 was linked with nephritis but not with any of the autoantibodies measured.

Treatment for SLE is directed at controlling the symptoms with the hope of putting the disease into remission. There are several chemotherapeutic agents in commercial use and available for remedial purposes. Most of these agents are not without side effects, some of which are severe and debilitating to the patient. Some non-steroidal anti-inflammatory agents may cause stomach upset and changes in kidney function, which can mimic some lupus symptoms themselves. Some anti-malarial drugs, when required at high dosage levels over a prolonged time frame, may accumulate in the retina and cause loss of vision. Certain steroidal preparations are used for their anti-inflammatory activity. The steroids, however, can exhibit side effects such as pronounced swelling of the face and abdomen, weight gain, excessive growth of body hair, cataracts, osteoporosis and heart attacks. Use of immunosuppressants can also have serious side effects such as changes in bone marrow, increased risk of infection to which the body normally shows resistance and a slight increase in the risk of developing certain types of cancer.

Another method of treatment for SLE, set forth in U.S. Pat. No. 4,690,905 to Diamond et al. generally describes generating monoclonal antibodies against anti-DNA antibodies (the monoclonal antibodies being referred to therein as anti-idiotypic antibodies) and then using these anti-idiotypic antibodies to remove the pathogenic anti-DNA antibodies from the patient's system. This approach, however, requires the removal of large quantities of blood for treatment in a process similar to hemodialysis. It is expensive and time-consuming, and is also associated with the risk of infection and/or hemorrhaging. Therefore, there remains a need for improved methods for diagnosing and treating SLE, as well as SLE-related diseases such as LN.

### SUMMARY OF THE INVENTION

The present invention is based on the identification of MDK as a genetic marker that is over-expressed in kidneys of mice having LN or predisposed for LN, relative to kidney samples from non-diseased controls. MDK is a heparin-binding growth factor that is known to be involved in tumorigenesis, angiogenesis and neural maturation/regeneration. The present invention provides a method for treating SLE/LN by restoring the activity of MDK or the expression of the MDK gene in diseased tissues to normal levels. The present invention further provides a method for diagnosing SLE/LN based on the expression level of MDK.

In one embodiment, the present invention provides a method for inhibiting MDK activity in a diseased tissue by a pharmaceutical composition comprising at least one of the following: (1) an agent that inhibits MDK activity, and (2) an agent that down-regulates MDK gene expression.

In another embodiment, the present invention provides methods for screening anti-lupus agents based on the agents' interaction with MDK, or the agents' effect on MDK gene expression or MDK activity.

The invention further provides cell lines harboring the MDK gene, animals transgenic for the MDK gene, and animals with an interrupted MDK gene (MDK knockout animals). These cell lines and animals can be used to study the functions of MDK.

In another embodiment, the present invention provides a method for diagnosing and monitoring SLE/LN by comparing the expression level of MDK at the nucleotide and/or protein level in biological samples from a subject to control samples.

In still another aspect, the invention provides polynucleotides capable of inhibiting MDK gene expression by RNA interference.

The invention further provides methods of inhibiting MDK gene expression by introducing siRNAs or other RNAi sequences into target cells.

The preferred embodiments of the inventions are described below in the Detailed Description of the Invention. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase.

It is further intended that the inventions not be limited only to the specific structure, material or methods that are described in the preferred embodiments, but in addition, include any and all structures, materials or methods that perform the claimed function, along with any and all known or later-developed equivalent structures, materials or methods for performing the claimed function.

Further examples exist throughout the disclosure, and it is not applicant's intention to exclude from the scope of the invention the use of structures, materials, or methods that are not expressly identified in the specification, but nonetheless are capable of performing a claimed function.

### BRIEF DESCRIPTION OF THE DRAWINGS

The inventions of this application are better understood in conjunction with the following drawings, in which:

FIG. 1 is a flow chart describing the steps for selecting LN-related genes.

FIG. 2 shows the gene expression frequency of MDK in LN-affected and control mice.

FIG. 3 shows the result of Taqman analysis of MDK expression in LN-affected and control mice.

FIG. 4 depicts MDK expression pattern in NZB×NZW F1 mice and the effect of rapamycin on MDK expression in these mice.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

The present invention is generally directed to compositions and methods for the diagnosis, treatment, and prevention of lupus, and to the identification of novel therapeutic agents for lupus. The present invention is based on the discovery of transcribed polynucleotides that are differentially expressed in animals that develop lupus or are pre-disposed to lupus.

Definitions and Terms

To facilitate the understanding of the present invention, a number of terms and phrases are defined below:

As used herein, a polynucleotide or a polypeptide is “isolated” if it is removed from its native environment. For instance, a polynucleotide or a polypeptide is isolated through a purification process such that the polynucleotide or polypeptide is substantially free of cellular material or free of chemical precursors. The polynucleotide/polypeptide of the present invention can be purified to homogeneity or other degrees of purity. The level of purification will be based on the intended use. As appreciated by one of ordinary skill in the art, a polynucleotide/polypeptide can perform its desired function(s) even in the presence of considerable amounts of other components or molecules.

In some uses, a polynucleotide/polypeptide that is “substantially free of cellular material” includes preparations which have less than about 30% (by weight) other polynucleotides/polypeptides including contaminating polynucleotides/polypeptides. For instance, the preparations can have less than about 20%, less than about 10%, or less than about 5% other polynucleotides/polypeptides. If a polynucleotide/polypeptide preparation is recombinantly produced, it can be substantially free of culture medium, i.e., culture medium components representing less than about 20% by weight of the polynucleotide/polypeptide preparation.

The language “substantially free of chemical precursors” includes preparations in which the polynucleotide/polypeptide is separated from chemical precursors or other chemicals that are involved in the synthesis of the polynucleotide/polypeptide. In one embodiment, the language “substantially free of chemical precursors” includes kinase preparations having less than about 30% (by weight), less than about 20% (by weight), less than about 10% (by weight), or less than about 5% (by weight) chemical precursors or other chemicals used in the synthesis.

A “polynucleotide” can include any number of nucleotides.. For instance, a polynucleotide can have at least 20, 25, 30, 40, 50, 100 or more nucleotides. A polynucleotide can be DNA or RNA, double-stranded or single-stranded. A polynucleotide encodes a polypeptide if the polypeptide is capable of being transcribed and/or translated from the polynucleotide. Transcriptional and/or translational regulatory sequences, such as promoter and/or enhancer(s), can be added to the polynucleotide before said transcription and/or translation occurs. Moreover, if the polynucleotide is singled-stranded, the corresponding double-stranded DNA containing the original polynucleotide and its complementary sequence can be prepared before said transcription and/or translation.

As used herein, “a variant of a polynucleotide” refers to a polynucleotide that differs from the original polynucleotide by one or more substitutions, additions, and/or deletions. For instance, a variant of a polynucleotide can have 1, 2, 5, 10, 15, 20, 25 or more nucleotide substitutions, additions or deletions. Preferably, the modification(s) is in-frame, i.e., the modified polynucleotide can be transcribed and translated to the original or intended stop codon. If the original polynucleotide encodes a polypeptide with a biological activity, the polypeptide encoded by a variant of the original polynucleotide variants substantially retains such activity.

Preferably, the biological activity is reduced/enhanced by less than 50%, or more preferably, less than 20%, relative to the original activity.

A variant of a polynucleotide can be a polynucleotide that is capable of hybridizing to the original polynucleotide, or the complementary sequence thereof, under reduced stringent conditions, preferably stringent conditions, or more preferably, highly stringent conditions. Examples of conditions of different stringency are listed in Table 1. Highly stringent conditions are those that are at least as stringent as conditions A-F; stringent conditions are at least as stringent as conditions G-L; and reduced stringency conditions are at least as stringent as conditions M-R. As used in Table 1, hybridization is carried out under a given hybridization condition for about 2 hours, followed by two 15-minute washes under the corresponding washing condition(s).

TABLE 1
Stringency Conditions

| Stringency | Polynucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | Tₚ*; 6xSSC | Tₚ*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, | | | | |
| # Tₘ(° C.) = 81 where N is the number of bases in the hybrid, and Na⁺ is the concentration of sodium ions in the hybridization buffer (Na⁺ for 1xSSC = 0.165M). | | | | |

It will be appreciated by those of ordinary skill in the art that, as a result of the degeneracy of the genetic code, there are many polynucleotide variants that encode the same polypeptide. Some of these polynucleotide variants bear minimal sequence homology to the original polynucleotide. Nonetheless, polynucleotides that vary due to differences in codon usage are specifically contemplated by the present invention.

As used herein, a “polypeptide” can include any number of amino acid residues. For instance, a polypeptide can have at least 5, 10, 15, 20, 30, 40, 50 or more amino acid residues.

As used herein, a “variant of a polypeptide” is a polypeptide that differs from the original polypeptide by one or more substitutions, deletions, and/or insertions. Preferably, these modifications do not substantially change (e.g. reduce or enhance) the original biological function of the polypeptide. For instance, a variant can reduce or enhance or maintain the biological activities of the original polypeptide. Preferably, the biological activities of the variant is reduced or enhanced by less than 50%, or more preferably, less than 20%, relative to the original polypeptide.

Similarly, the ability of a variant to react with antigen-specific antisera is preferably enhanced or reduced by less than 50%, preferably less than 20%, relative to the original polypeptide. These variants can be prepared and evaluated by modifying the original polypeptide sequence and then determining the reactivity of the modified polypeptide with the antigen-specific antibodies or antisera.

Preferably, a variant polypeptide contains one or more conservative substitutions. A “conservative substitution” is one in which an amino acid is substituted for another amino acid which has similar properties, such that one skilled in the art would expect that the secondary structure and hydropathic nature of the substituted polypeptide will not be substantially changed. Conservative amino acid substitutions can be made on the basis of similarity in polarity, charge, solubility, hydrophobicity, hydrophilicity and/or the amphipathic nature of the residues. Negatively charged amino acids include aspartic acid and glutamic acid, and positively charged amino acids include lysine and arginine. Amino acids having uncharged polar head groups and similar hydrophilicity values include leucine, isoleucine and valine, or glycine and alanine, or asparagine and glutamine, or serine, threonine, phenylalanine and tyrosine. Other groups of amino acids that can produce conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A polypeptide variant can also contain nonconservative changes.

Polypeptide variants can be prepared by substituting, modifying, deleting and/or adding one or more amino acids that have minimal influence on the biological activity, immunogenicity, secondary structure and/or hydropathic nature of the polypeptide. Variants can be prepared by substituting, deleting or adding, for example, 1, 2, 5, or 10 amino acids residues in the original sequence. Polypeptide variants preferably exhibit at least about 70%, more preferably at least about 90%, and most preferably at least about 95% sequence homology to the original polypeptide.

Polypeptide variants include polypeptides that are modified from the original polypeptides either by a natural process, such as a post-translational modification, or by a chemical modification. These modifications are well known in the art. Modifications can occur anywhere in the polypeptide, including the backbone, the amino acid side-chains and the amino or carboxyl termini. It will be appreciated that the same type of modification can be present in the same or varying degrees at several sites in a given polypeptide. Also, a given polypeptide can contain many types of modifications. Polypeptides may be branched, for example, as a result of ubiquitination, and they may be cyclic, with or without branching. Cyclic, branched, and branched cyclic polypeptides can result from natural post-translational processes or be made through synthetic methods. Suitable modifications for this invention include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

As used herein, the term “modulation” includes up-regulation, induction, stimulation, potentiation, inhibition, down-regulation or suppression, or relief of inhibition.

A nucleotide sequence is “operably linked” to another nucleotide sequence if the two sequences are placed into a functional relationship. For example, a coding sequence is operably linked to a 5′ regulatory sequence if the 5′ regulatory sequence can initiate transcription of the coding sequence in an in vitro transcription/translation system or in a host cell. “Operably linked” does not require that the DNA sequences that are linked are contiguous to each other. Intervening sequences may exist between two operably linked sequences.

A polynucleotide is “capable of hybridizing” to a gene if the polynucleotide can hybridize to at least one of the following sequences: (1) the sequence of a RNA transcript of the gene, (2) the complementary sequence of a RNA transcript of the gene, (3) the cDNA sequence of a RNA transcript of the gene, (4) the complementary sequence of the cDNA sequence of a RNA transcript of the gene, (5) a genomic sequence of the gene, and (6) the complementary sequence of a genomic sequence of the gene.

As used herein, the term “antigen-specific” refers to antibodies that bind to the antigen of interest (e.g., MDK or homologs thereof or a fragment) with an affinity equal to, or greater than, 10⁵M⁻¹.

As used herein, the term “normal” refers to cells, tissues or other such samples taken either pre-disorder or from a subject who has not suffered LN or SLE, or from a cell, tissue or sample that is substantially free of LN and SLE. Control samples of the present invention are taken from normal samples.

As used herein, the terms “treating,” “treatment,” and “therapy” as used herein refer to curative therapy, prophylactic therapy, and preventative therapy.

As used herein, sequence identity or percentage alignment can be determined by the standard protein-protein or nucleotide-nucleotide BLAST programs (i.e., blastp or blastn, respectively). Sequence identity or percentage alignment can also be determined by the BLAST2 program. Suitable BLAST programs can be found at the BLAST web site maintained by the National Center of Biotechnology Information (NCBI) (National Library of Medicine, USA).

Various aspects of the invention are described in further detail in the following subsections. The use of subsections is not meant to limit the invention, but rather the subsections may apply to any aspect of the invention.

Midkine and LN

The gene expression pattern in kidneys of 4 different strains of mice: MRL/MpJ-Fas^{lpr}, MRL/MpJ, C57B16 and C57B16/Fas^{lpr}, were evaluated using the Affymetrix Mu11KsubA and Mu11KsubB oligonucleotide arrays (Affymetrix, Santa Clara, Calif.).

Briefly, the gene expression analysis was performed using kidney RNA samples harvested from individual mice. The RNA samples were reverse transcribed into cDNA and hybridized to the oligonucleotide arrays. The results were analyzed using Microarray Suite software. A gene analysis set of 5285 oligonucleotides was first selected using the criteria described in Example 3. The expression frequency of each gene on the 5285 oligonucleotides in the gene analysis set was then determined for all C57B16, C57B16/Fas^{lpr}, MRI-MpJ-Fas^{lpr} and MRL/MpJ kidney samples (n=46). In order to identify gene expression patterns that may contribute disease initiation, selected first were genes with significantly different expression levels in young, pre-symptomatic MRL/MpJ kidney and kidneys from mice that do not develop LN. Late stage disease samples (i.e, samples from MRL/MpJ-Fas^{lpr} mice four months of age or older) were omitted from this initial screen due to the numerous and profound changes in gene expression related to inflammation, kidney failure and fibrosis observed at this stage of disease. These changes are known consequences of the disease process, and would be expected to obscure differences identified between disease free and early stage disease samples.

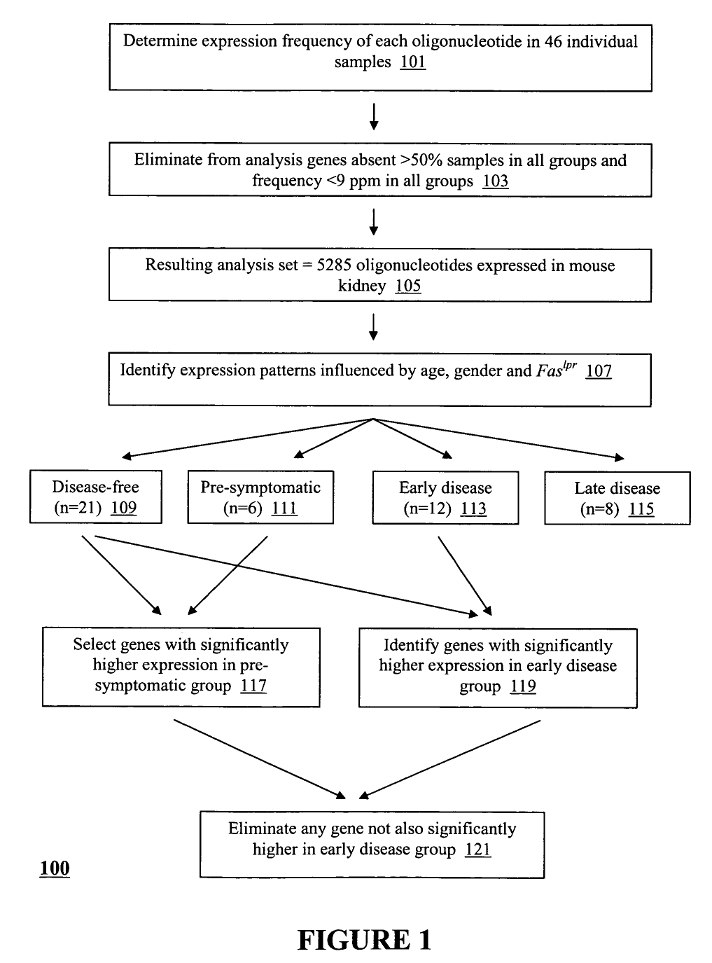

FIG. 1 shows a flow chart describing the process 100 for selecting LN-related genes. Defining significant difference between groups as p<0.0005 (two tailed student t test, unequal variance) and average fold change (AFC)>1.5, a list of genes with significant expression frequency differences between lupus nephritis negative samples (C57BL/6, C57BL6/Fas^{lpr}) and young (pre-symptomatic) MRL/MpJ-Fas^{lpr} kidneys was compiled (step 101). Genes on this list that did not also show significant expression level differences (again defined as p<0.0005, AFC>1.5) between lupus nephritis negative samples and early stage disease samples (i.e., samples consisting of the 6 older MRL/MpJ and the 6 young MRL/MpJ-Fas^{lpr} samples) were removed from the list (step 103). This step was taken to eliminate any genes whose expression patterns failed to track with disease progression. The gene expression patterns influenced by age, gender and Fas^{lpr} was then identified using the resulting gene analysis set of 5285 oligonucleotides in all kidney samples (steps 105-115). Genes with significantly higher expression in pre-symptomatic group and early disease group are then identified (steps 117 and 119). Finally, only those genes that have significantly higher expression in both groups are selected for further analysis (step 121).

Fourteen full length sequences, 12 for genes with known function, and 9 ESTs comprise the list of oligonucleotides with significantly higher expression levels in MRL/MpJ than C57B16 kidneys. The genes on the list were not among those flagged as demonstrating age, gender or Fas^{lpr} dependent expression patterns (identified as described in Example 4), indicating that the selection process successfully eliminated any gene expression patterns dependent on these factors.

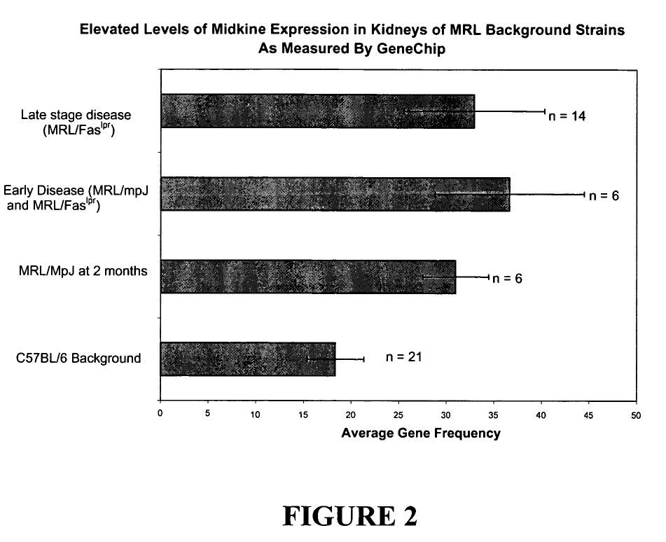

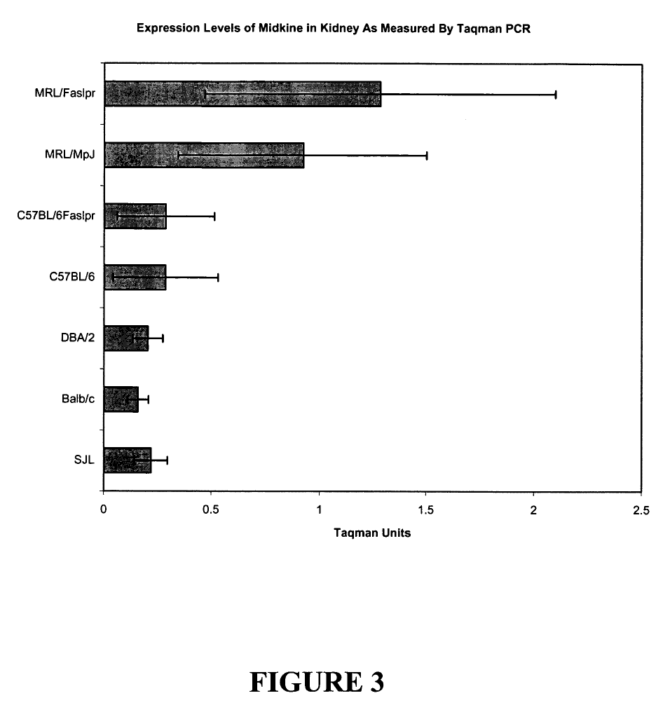

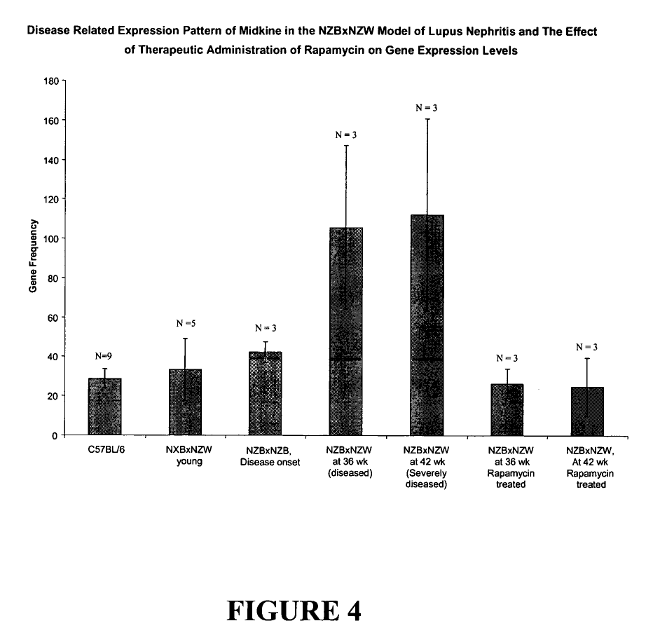

Among the genes of known function on the list, MDK is a promising candidate for developing diagnosis methods and therapeutics to SLE/LN. As shown in FIG. 2, the gene expression frequency of MDK in LN-affected mice (i.e., MRL/MpJ-Fas^{lpr} mice at two and five months of age, and MRL/MpJ at two months of age) was significantly higher than that in control mice (C57BL/6 and C57BL/6-Fas^{lpr} combined). The elevated MDK expression in LN-affected mice was confirmed by Taqman PCR analysis. As shown in FIG. 3, the midkine expression levels in kidneys of DBA/2, SJL and BALB/c mice are similar to those in C57BL/6 and C57BL/6-Fas^{lpr} mice, but are significantly lower than the levels in MRL/MpJ and MRL/MpJ-Fas^{lpr} mice. Further studies revealed that MDK is also over-expressed in the kidney of lupus-affected NZB×NZW F1 mice, and that therapeutic administration of rapamycin in the diseased NZB×NZW F1 mice restored the MDK expression to normal levels (FIG. 4). Taken together, these data strongly suggest that the elevated MDK expression is closely associated with lupus development in the kidney of LN-affected mice and that the successful treatment of LN involves restoring normal MDK levels.

The Biochemical and Biological Characteristics of MDK

MDK is a secreted protein that is expressed in a wide range of cell types and tissues at certain stages of development, and especially in neuron-glial cells and tumor cells. The biological functions of MDK have been studied extensively.

(1) Function in Neurone-glial Interaction

MDK expression during development is temporally and spatially regulated. The patterns of expression suggest that MDK plays a role in neural maturation, epithelial/mesenchyme interactions and secondary embryonic induction processes. Generally, MDK expression in the central nerve system (CNS) occurs early in embryogenesis and is completed by birth. MDK expression is barely detectable in adults except in kidney and certain CNS areas. Immunohistochemical studies revealed MDK to be localized beside radial glial processes along which neurons migrate. It has been suggested that MDK is synthesized by and localized on the surface of radial glial cells, while its receptor system is localized on neurons (Sun et al., J. Neuropathol Exp Neurol 56:1339-1348, 1997). Upon experimental infraction in rats, MDK expression in the isochemic brain begins as early as 1 day after the operation. MDK expression is also induced in Alzheimer's senile plaque and in photoreceptor cells rescued from light-induced damage, suggesting involvement of MDK in repair and regeneration mechanisms in the nervous system. MDK has also been reported to promote neuronal survival in culture, to stimulate neuronal differentiation, and to be involved in synaptogenesis. As MDK is expressed in glial cells, most notably in the radial glia of the neocortex and the Bergmann fibres of the cerebellar cortex, it may also have a role in the migration of neuron progenitors prior to the onset of axon navigation.

(2) Function in Inflammatory Responses

MDK has also been detected in synovial fluid, synoviocytes, and endothelial cells of new blood vessels in the inflammatory synovitis of rheumatoid arthritis and osteoarthritis, but was not detected in normal synovial fluid and non-inflammatory synovial tissues. MDK promotes chemotaxis of neutrophils and histamine release from rat peritoneal mast cells in a dose-dependent manner. MDK also enhances plasminogen activator activity and reduces plasminogen activator inhibitor levels in bovine aortic endothelial cells. These activities of MDK are in agreement with the modes of MDK expression in various pathological states. It has thus been suggested that MDK is an important molecule regulating inflammatory responses.

(3) Function in Tumorigenesis and Angiogenesis

MDK influences cell growth both in vitro and in vivo. It enhances neurite outgrowth extension on PC12 cells and primary neuronal cells from rat- or chicken-sympathetic neurons, and survival of mesencephalic neurons and embryonic day 12 chicken sympathetic neurons. Native mouse MDK protein tested on pig EC cells induced cell differentiation. MDK is also mitogenically active on PC12 cells, 10T1/2 fibroblasts, neuroectodermal precursor cells for immature 1009 EC cells, and NIH 373 fibroblasts.

MDK purified from the conditioned media of MDK transfected SW-13 cells stimulated colony formation of the parent SW-13 cells as well as proliferation of human brain and umbilical vein endothelial cells. Furthermore, SW-13 cells expressing high levels of MDK grew into tumors in nude mice. Similar tumor growth was reported for NIH 3T3 cells after expression of a human MDK cDNA (Kadomatsu et al., Br J Cancer 75:354-359, 1997). Overexpression of exogenous MDK in MCF-7 breast carcinoma cells had no effect on in vitro growth but conferred a growth advantage in vivo. Enhanced tumor growth correlated with increased vascular density and endothelial proliferation, implicating an angiogenic role for MDK. Angiogenic activity of MDK was also confirmed in a rabbit corneal assay (Choudhuri et al., Cancer Res 75:354-359, 1997).

MDK is overexpressed in a variety of malignant tumor cells, including hepatocellular carcinoma (HCC), colorectal cancer, gastric cancer, esophageal cancer, lung cancer, breast cancer, ovarian cancer, bladder cancer, pancreatic duct adenocarcinoma, Wilm's tumor, thyroid papillary carcinoma, neuroblastoma, neurofibroma, astrotoma, brain tumor and embryonal carcinoma cells. In addition, the overexpression rate of MDK protein in HCC with intra-hepatic metastasis was significantly higher than that in HCC without intra-hepatic metastasis. It thus appears that MDK may be closely related to local infiltration and metastasis of human hepatocellular carcinoma. More interestingly, a truncated form of MDK mRNA that lacks a sequence encoding the N-terminally located domain was found in cancer cells and resected specimens of human breast cancer, gastrointestinal cancer, colorectal carcinomas, Wilm's tumor, hepatocellular carcinomas and capillary duodenal cancer, but not in non-cancerous tissues. This data strongly suggests that MDK plays a significant role in tumorigenesis and angiogenesis, and may be used as a marker of diagnosis and a targeting candidate of malignant cancer therapy. Since MDK is a secreted protein, it can be easily detected in body fluid samples, such as urine and blood. Antisense oligodeoxynucleotide target to MDK has been found to suppress tumorigenicity of rectal carcinoma cells in nude mice (Takei et al., Cancer Res 61:8486-8491, 2001; Takei et al., J Biol Chem 2002).

(4) MDK's Involvement in Signal Pathways

Ligand homodimerization appears to be the initial step in the activation of a common signal pathway for MDK. Dimer formation through transglutaminase-mediated cross-linking is important for the biological activity of MDK. Proteoglycan-binding enhances the biological activities of MDK in a manner analogous to members of the fibroblast growth factor family. Covalently bound homodimers are uncommon amongst tyrosine kinase receptor ligands. In the case of MDK, there is evidence that stable non-disulphide bonded dimers are formed through the catalytic activity of type-2 transglutaminase, an enzyme which crosslinks neural substrates through ε-(δ-glutamyl)lysine isopeptide bonds. Upon incubation with transglutaminase, MDK forms multimers through cross-linkages. It was found that (1) heparin potentiated the multimer formation; (2) the N- and C-terminal half domains each formed a dimer through the action of transglutaminase; (3) Gln42 or Gln44 in the N-terminal half and Gln95 in the C-terminal half served as amine acceptors in the cross-linking reaction; and (4) MDK-derived peptide Ala41-Pro51 strongly inhibited the cross-linking and abolished the biological activity of MDK to enhance the plasminogen activator activity in bovine aortic endothelial cells. The inhibition, however, was limited against the MDK monomer and was not seen against the MDK dimer, suggesting that dimer formation through transglutaminase-mediated cross-linking is an important step affecting the biological activity of MDK. This notion is further supported by the finding that the interaction of dimeric MDK with corresponding glycosaminoglycan binding sites is the basis for cooperation of proteoglycans in signaling. Since the isopeptide bond is highly resistant to degradation, this novel means of covalent association potentiates signaling.

It is likely that two or more high-affinity tyrosine kinase receptors exist for MDK. MDK forms dimers before associating with its receptors and appears to activate tyrosine kinase, JAK/STAT-1 and PI 3-kinase signal pathways. These properties suggest that MDK is a ligand for tyrosine kinase receptors. MDK also interacts strongly with cell surfaces, and its binding sites include proteoglycans. Studies to date on the signal pathways of MDK suggest that its common cell surface binding domains in neurons have at least three interactive components: syndecan-3/proteoglycan complexes, the receptor-like protein tyrosine phosphatase-ζ and contactin. In addition, the highly basic C- and N-terminal domains of both proteins interact with charged “docking sites” at the cell surface. These may be identical to one of the above components, or may be a separate glycoprotein or glycolipid. Sulfatide and other glycosphingolipids bearing neolacto-glycoside sidechains bind both MDK and amphoterin, a larger relation to the MDK family, and currently are candidate molecules for the docking site. An LDL receptor-related protein may also function as a part of a MDK receptor complex at the plasma membrane of cells.

(5) Anti-apoptotic Activities

MDK inhibits apoptosis via extracellular signal-regulated kinase (ERK) activation in an apoptosis induction system using primary neuronal cultures isolated from mouse cerebral cortices. In this system, neuronal apoptosis induced by serum deprivation was accompanied by the activation of caspase-3. MDK inhibited the induction of apoptosis and activation of caspase-3 in a dose-dependent manner. Extracellular signal-regulated kinase (ERK) and Akt were not activated by serum deprivation, but were rapidly activated by addition of MDK. The trophic actions of MDK of suppressing apoptosis and suppressing the activation of caspase-3 were abolished by concomitant treatment with PD98059, a specific inhibitor of mitogen-activated protein kinase, and with wort-mannin or LY294002, specific inhibitors of phosphatidyl-inositol 3-kinase (PI 3-kinase). These PI 3-kinase inhibitors also inhibited the activation of ERK in response to MDK, demonstrating a link between ERK and the caspase-3 pathway that is modulated by the PI 3-kinase activation. These results indicate that the ERK cascade plays a central role in MDK-mediated neuronal survival via inhibition of caspase-3 activation.

The neuroprotective actions of MDK via ERK activation is further demonstrated in PC12 cells. Specifically, MDK rescued PC 12 cells from apoptosis induced by serum deprivation in a dose-dependent manner. In agreement with the earlier findings, MDK activated ERK1 and ERK2; and PD98059 inhibited ERK activation and also prevented the trophic effect of MDK.

MDK also rescues Wilms' tumor cells from cisplatin-induced apoptosis. Cisplatin (CDDP), a chemotherapy drug, induces recoverable renal damage and apoptosis in the kidney of adult mice. In vivo, cisplatin transiently suppressed MDK expression in mouse kidney. In vitro, CDDP suppressed MDK expression and induced apoptosis in cultured G401 cells, a Wilms' tumor cell line. However, exogenous MDK protein partially rescued G401 cells from CDDP-induced apoptosis. It was found that MDK enhanced the expression of Bcl-2, but not that of Bcl-x(L), in G401 cells in a dose-dependent manner. MDK also prevented the Bcl-2 reduction due to CDDP. Moreover, Bcl-2 expression in mouse kidney was also transiently suppressed by CDDP treatment, the expression profile being similar to that of MDK. It thus appears that MDK exerts cytoprotective activity toward a damaging insult, presumably at least in part through enhancement of the expression of Bcl-2.

(6) MDK Protein Structure

Human MDK is a secreted glycoprotein with a molecular mass of about 13 kD. Human MDK precursor has 143 amino acid residues (SEQ ID NO:1), including a 22-amino acid leader peptide (SEQ ID NO:2). Mature MDK (SEQ ID NO:3) is structurally divided into two domains, an N-terminal domain and a C-terminal domain. The solution structure of the two domains was determined by NMR (Iwasake et al., EMBO J 16:6936-6946, 1997). Both domains consist of three antiparallel beta-sheets, but the C-terminal domain has a long flexible hairpin loop where a heparin-binding consensus sequence is located. Basic residues on the beta-sheet of the C-terminal domain form another heparin-binding site. Measurement of NMR signals in the presence of heparin oligosaccharides verified that multiple amino acids in the two sites participated in heparin binding.

A 121-amino-acid-residue human MDK (SEQ ID NO:3) has been synthesized in solution (Peptide Institute Inc., J Pept Sci 2:28-39, 1996). The final product was confirmed to have the correct disulphide structure from its tryptic peptide mapping and to possess the same biological activities as those of the natural product. The N- and C-terminal domains [(1-59aa) and (60-121 aa), respectively] were also synthesized. The C-terminal domain showed the full pattern of bioactivities except for the neuronal cell survival activity, while the N-terminal domain had much less activity in general (supra). The 13 amino acid residues in the C-terminal end were found to be responsible for the MDK antigenicity (Muramatsu et al., Biochem Biophys Res Commun 203:1131-1139, 1994).

MDK is also characterized by a high content (e.g., about 25%) of basic amino acid residues, predominately lysine, which result in the proteins having high pI values of around 10. The basic residues are not evenly distributed throughout the polypeptide chain, but are clustered at the N-terminus and at the C-terminus with another concentration in the C-domain. The rule of these basic residues on the receptor binding and biological actions of MDK is currently under study. The two clusters of basic residues in the C-terminal part of MDK are vital for interaction with heparin as shown by NMR spectroscopy (Iwasake et al., EMBO J 16:6936-6946, 1997). The removal of N-terminally located clusters of basic amino acids (N-tail) or C-terminally located clusters of basic amino acids (C-tail) from the MDK molecule severely reduced its neurite-promoting activity. However, experiments involving chemically synthesized MDK derivatives revealed that the roles of the N-tail and C-tail were mostly indirect ones, i.e., they probably maintain the steric arrangements of the N-terminal and C-terminal halves. In particular, the C-domain, which is the C-terminal half devoid of the C-tail, retained considerable neurite-promoting activity when it was uniformly coated on a dish. The removal of the N-tail or C-tail also reduced the enhancing activity of plasminogen activator (PA) in aortic endothelial cells, although the effect was lower. There are two heparin-binding sites in the C-domain, Clusters I and II. A mutation in Cluster I [R78-->Q] affected the PA-enhancing activity only slightly, and a mutation in Cluster II [K83K84-->QQ] abolished the activity, while both mutations are known to reduce the neurite-promoting activity moderately. Therefore, the two heparin-binding sites in the C-domain play different roles in these two activities. Indeed, heparin exhibited different effects on these two activities. It was also observed that intact MDK was required for ordered neurite-promotion along the path of MDK. One possible interpretation of this is that the N-terminal half is necessary for the stability of the molecule. Furthermore, K76 and K99 were found to be required for the secretion of MDK, i.e., mutants in which one of these K residues was changed to Q were produced in the host cells, but not found in the medium.

The heparin-binding domain of MDK has been studied extensively. The three dimensional structure of MDK clarified by NMR spectroscopy indicates that several basic amino acids are exposed on the surface of the C-terminal half domain, which retains heparin-binding and neurite-promoting activity. Site-directed mutagenesis revealed that mutation of arginine78 reduced the heparin-binding activity. Mutation of either lys83 or lys84 scarcely affected heparin-binding activity, while the double mutant involving both lysine residues showed reduction in the activity (Asai et al., Biochem Biophys Res Commun 236:66-70, 1997). Neurite-promoting activity of mutant MDKs always correlated with their heparin-binding activity, illustrating the close relationship of the two activities.

The inhibitory activities of various heparin derivatives toward interaction of MDK with neurons were also examined (Kaneda et al., Biochem Biophys Res Commun 220:108-112, 1996). All of the three sulfate groups in the heparin disaccharide unit (6-O-sulfate, 2-O-sulfate and N-sulfate) were necessary for full inhibitory activity. Among these, the N-sulfate group was critically important. The minimum size with inhibitory activity was approximately 7 kd. Thus, the highly sulfated region in cell surface heparin sulfate proteoglycan is required for neurons to interact with MDK.

(7) MDK Gene and MDK Promoter

Human MDK gene (SEQ ID NO:4) contains five exons and four introns with the coding sequences present in exons 2-5. The MDK gene is located on chromosome 11q11.2. MDK gene sequences from different species share a high level of homology. For example, human and mouse MDK is 87% identical at amino acid level and most amino acid changes are conservative. All the characteristic cysteine and lysine residues are conserved. This high degree of evolutional conservation reinforces the important role of MDK during embryogenesis. The organization of the human MDK gene is also similar to that of the mouse MDK gene. All exon-intron boundaries are conserved between mouse and human MDK. It was found that a 170 base block in the upstream region of the putative transcription initiation sites and three blocks of 200-350 bases in regions further upstream are highly conserved. These homologous blocks may play important roles in developmentally-regulated expression of the MDK gene. Further analysis revealed that the 2.3 kb upsteam sequence of the human MDK gene has cis-acting elements which confer retinoic acid-induced expression of a fused chloramphenicol acetyl-transferase (CAT) gene in F9 embryonal carcinoma cells. In the 5′-region of the human MDK gene, a sequence resembling the DR5-type retinoic acid-responsive element (i.e., AGGTCA-related direct repeats separated by 5 nucleotides) is present in a small block that is highly homologous between the human and mouse genes. Deletion of this direct repeat reduced retinoic acid-induced CAT gene expression (Pedraza et al., J Biochem 117:845-849, 1995). Because the MDK promoter appears to be active only in tumor tissues, the human MDK promoter has been used in adenoviral suicide gene therapy for pancreatic cancer and MDK-positive pediatric tumor.

Polymerase chain reaction-single strand conformation polymorphism (PCR-SSCP) analysis revealed an association of an intronic polymorphism in the MDK gene with human sporadic colorectal cancer (Ahmed et al., Cancer Lett 180:159-63, 2002). Gene alterations were also found in the MDK promoter region in some sporadic colorectal and gastric cancer patients (Ahmed et al., Int J Mol Med 6:281-287, 2000).

(8) MDK Family

A number of proteins with strong homology to MDK have been reported. Among them is pleiotrophin (PTN). MDK and PTN have about 50% sequence identity at the amino acid level, and all cysteine residues are conserved. The functions of MDK and PTN are similar. Other members of the family include retinoic acid-induced heparin-binding protein (RIHB) (Urios et al., Biochem Biophys Res Commun 175:617-624, 1991) and amphoterin (Nair et al., Neuroscience 85:759-771, 1998).

In summary, the biochemical and biological characteristics of MDK not only indicate its biological significance in transforming, anti-apoptotic, angiogenic, fibrinolytic activities, but also support its possible involvement in the development of auto-immune diseases such as lupus. The current understanding on MDK protein structure and function facilitate the clinical application of MDK in the diagnosis and treatment of lupus.

MDK as a Marker for SLE and LN

MDK has not been previously associated with SLE and LN. The present invention identifies MDK as a marker of SLE and LN, which is differentially expressed in kidneys of LN-affected MRL/MpJ-Fas^{lpr}, MRL/MpJ, and NZB×NZW F1 mice, relative to kidney samples from control C57BL/6 and C57BL/6-Fas^{lpr} mice. The marker can be a component in the disease mechanism is a novel therapeutic target for the treatment and prevention of SLE/LN. While mouse models were used for the initial differentiation expression analysis, it is well-appreciated in the art that expression levels of genes in animal models can be interpreted to reflect expression levels from human subjects as well. The present invention and understood that the present invention specifically encompasses human MDK. MDK homologs from other organisms may also be useful in the use of animal models for the study of SLE and LN and for drug evaluation. MDK homologs from other organisms may be obtained using the techniques outlined below.

Accordingly, the present invention pertains to the use of MDK gene, the transcribed polynucleotides, and the encoded polypeptides as markers for SLE/LN. For example, MDK gene or gene fragments can be conveniently arrayed on solid supports, i.e., biochips, such as the GeneChip®, as probes to detect MDK mRNA. Anti-MDK antibody can be developed and used in diagnostic kits to detect MDK protein levels in body fluids. The markers can be used to provide diagnosis or prognosis information in a particular subject sample or to assess the efficacy of a treatment or therapy of SLE/LN. For example, comparison of expression levels of MDK at different stages of the disease progression provides a method for long-term prognosing, including survival. MDK gene polymorphism may also be indicative to a subject's susceptibility to SLE/LN. In another example, the evaluation of a particular treatment regime may be evaluated, including whether a particular drug will act to improve the long-term prognosis in a particular patient.

MDK promoter, MDK gene, and MDK gene products (the transcribed polynucleotides and the translated polypeptides) can be targets for a treatment or therapeutic agent. They can also be used to generate gene therapy vectors that inhibit lupus.

Therefore, without limitation as to mechanism, the present invention is based in part on the principle that modulation of the expression of the MDK gene expression may ameliorate SLE/LN, when they are expressed at levels similar or substantially similar to normal (non-diseased) tissue. The modulation may occur at transcriptional, post-transcriptional, translational, and post-translational levels. For example, MDK promoter may be targeted to inhibit transcription. MDK mRNA may be targeted by anti-sense molecules to prevent translation. The post-translational processing of MDK protein, such as leader peptide removal, glycosylation and dimerization, may also be targeted.

The discovery of the MDK gene expression patterns in SLE/LN affected animal allows for screening of test agents with the goal of modulating MDK expression or MDK activity. The test agents may be screened by their effect on MDK expression at mRNA or protein level, or by their effect on the activity of MDK.

In another embodiment of the invention, a modulator of MDK expression or MDK activity may be used as a therapeutic agent for SLE and LN. The modulator may be a polynucleotide such as an antisense oligonucleotide, a polypeptide such as an anti-MDK antibody or a MDK mutant having a dorminant negative effect on a activity of the wild-type MDK, a viral or non-viral gene therapy vector, or any other organic or inorganic molecule that is capable of inhibiting MDK activity or MDK expression. Formulation of such modulator into pharmaceutical compositions is described in subsections below.

Isolated Polynucleotides

One aspect of the invention pertains to isolated polynucleotide fragments sufficient for use as hybridization probes to identify the MDK gene or MDK gene products in a sample, as well as nucleotide fragments for use as PCR primers of the amplification or mutation of the nucleic acid molecules which encode MDK. Another aspect of the invention pertains to isolated polynucleotides that encode MDK, a fragment of MDK or a mutant of MDK.

A polynucleotide comprising the nucleotide sequence of MDK (SEQ ID NO:4), or homologs thereof, or a portion thereof, can be isolated using standard molecular biology techniques and the sequence information provided herein as well as sequence information known in the art. Using all or a portion of the polynucleotide sequence of MDK (or a homolog thereof) as a hybridization probe, a MDK gene or a polynucleotide transcribed from a MDK gene can be isolated using standard hybridization and cloning techniques.

A MDK gene can be amplified using cDNA, mRNA or alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to MDK gene nucleotide sequences can be prepared by standard synthetic techniques, e.g., using an automated DNA synthesizer.

In another preferred embodiment, an isolated polynucleotide of the invention comprises a polynucleotide which is a complement of the nucleotide sequence of a MDK gene, or homolog thereof, a polynucleotide transcribed thereof, or a portion of any of these nucleotide sequences. A polynucleotide which is complementary to such a nucleotide sequence is one which is sufficiently complementary to the nucleotide sequence such that it can hybridize to the nucleotide sequence, thereby forming a stable duplex.

The polynucleotide of the invention, moreover, can comprise only a portion of the polynucleotide sequence of a MDK gene, for example, a fragment which can be used as a probe or primer. The probe/primer typically comprises substantially purified oligonucleotide. The oligonucleotide typically comprises a region of nucleotide sequence that hybridizes under stringent conditions to at least about 7 or 15, preferably about or 25, more preferably about 50, 75, 100, 125, 150, 175, 200, 225, 250, 275, 300, 325, 350, 400 or more consecutive nucleotides of a MDK gene, or a polynucleotide transcribed thereof.

Probes based on the nucleotide sequence of a MDK gene, or a polynucleotide transcribed thereof can be used to detect transcripts or genomic sequences corresponding to the MDK gene, or a polynucleotide transcribed thereof. In preferred embodiments, the probe comprises a label group attached thereto, e.g., the label group can be a radioisotope, a fluorescent compound, an enzyme, or an enzyme co-factor. Such probes can be used as a part of a diagnostic test kit for identifying cells or tissue which mis-express (e.g., over- or under-express) a MDK gene, or which have greater or fewer copies of a MDK gene. For example, a level of a MDK gene product in a sample of cells from a subject may be detected, the amount of mRNA transcript of MDK may be determined, or the presence of mutations or deletions of a MDK gene may be assessed.

The invention also specifically encompasses homologs of the MDK gene of other species. Gene homologs are well understood in the art and are available using databases or search engines such as the Pubmed-Entrez database.

The invention also encompasses polynucleotides that are structurally different from the molecules described above (i.e., which have a slight altered sequence), but which have substantially the same properties as those above (e.g., encoded amino acid sequences, or which are changed only in non-essential amino acid residues). Such molecules include allelic variants, and are described in greater detail in subsections herein.

In addition to the nucleotide sequences of the MDK gene, it will be appreciated by those of skill in the art that DNA sequence polymorphisms that lead to changes in the amino acid sequences of the proteins encoded by the MDK gene may exist within a population (e.g., the human population). Such genetic polymorphism in the MDK gene may exist among individuals within a population due to natural allelic variation. An allele is one of a group of genes which occur alternatively at a given genetic locus. In addition it will be appreciated that DNA polymorphisms that affect RNA expression levels can also exist that may affect the overall expression level of that gene (e.g., by affecting regulation or degradation). As used herein, the phrase “allelic variant” includes a nucleotide sequence which occurs at a given locus or to a polypeptide encoded by the nucleotide sequence.

Polynucleotides corresponding to natural allelic variants and homologs of the MDK gene can be isolated based on their homology to the human MDK gene, using the cDNAs disclosed herein (SEQ ID NO:4), or a portion thereof, as a hybridization probe according to standard hybridization techniques under stringent hybridization conditions. Polynucleotides corresponding to natural allelic variants and homologs of the MDK gene can further be isolated by mapping to the same chromosome or locus.

In another embodiment, an isolated polynucleotide of the invention is at least 15, 20, 25, 30, 50, 100, 150, 200, 250, 300, 350, 400, or more nucleotides in length and hybridizes under stringent conditions to a polynucleotide corresponding to a nucleotide sequence of a MDK gene. Preferably, an isolated polynucleotide of the invention that hybridizes under stringent conditions to the sequence of a MDK gene corresponds to a naturally-occurring polynucleotide.

In addition to naturally-occurring allelic variants of a MDK gene that may exist in the population, the skilled artisan will further appreciate that changes can be introduced by mutation into the nucleotide sequences of the MDK gene, thereby leading to changes in the amino acid sequence of the encoded proteins, without altering the functional activity of these proteins. For example, nucleotide substitutions leading to amino acid substitutions at “non-essential” amino acid residues can be made. A “non-essential” amino acid residue is a residue that can be altered from the wild-type sequence of a protein without altering the biological activity, whereas an “essential” amino acid residue is required for biological activity. For example, amino acid residues that are conserved among allelic variants or homologs of a gene (e.g., among homologs of a gene from different species) are predicted to be particularly unamenable to alteration.

Accordingly, another aspect of the invention pertains to polynucleotides encoding MDK proteins that contain changes in amino acid residues that are not essential for activity. Such proteins differ in amino acid sequence from the original MDK protein encoded by the MDK gene, yet retain biological activity. In one embodiment, the protein comprises an amino acid sequence at least about 60%, 65%, 70%, 75%, 80%, 85%, 90%, 95%, 98% or more homologous to a MDK protein.

In yet other aspect of the invention, polynucleotides of a MDK gene may comprise one or more mutations. An isolated polynucleotide encoding a protein with a mutation can be created by introducing one or more nucleotide substitutions, additions or deletions into the nucleotide sequence of the polynucleotide, such that one or more amino acid substitutions, additions or deletions are introduced into the encoded protein. Such techniques are well known in the art. Mutations can be introduced into a MDK gene by standard techniques, such as site-directed mutagenesis and PCR-mediated mutagenesis. Preferably, conservative amino acid substitutions are made at one or more predicted non-essential amino acid residues. Alternatively, mutations can be introduced randomly along all or part of a coding sequence of the MDK gene or cDNA, such as by saturation mutagenesis, and the resultant mutants can be screened for biological activity to identify mutants that retain activity. Following mutagenesis, the encoded protein can be expressed recombinantly and the activity of the protein can be determined.

In yet another aspect of the invention, a polynucleotide may encode a MDK protein containing mutations in amino acid residues which result in inhibition of MDK activity after dimerization with a wild-type MDK protein. These mutated MDK proteins can be used to inhibit MDK activity in a SLE/LN patient.

A polynucleotide of this invention can be further modified to increase stability in vivo. Possible modifications include, but are not limited to, the addition of flanking sequences at the 5′ and/or 3′ ends; the use of phosphorothioate or 2-o-methyl rather than phosphodiester linkages in the backbone; and/or the inclusion of nontraditional bases such as inosine, queosine and wybutosine, as well as acetyl- methyl-, thio- and other modified forms of adenine, cytidine, guanine, thymine and uridine.

Another aspect of the invention pertains to isolated polynucleotides, which are antisense to a MDK gene. An “antisense” polynucleotide comprises a nucleotide sequence which is complementary to a “sense” polynucleotide encoding a protein, e.g., complementary to the coding strand of a double-stranded cDNA molecule or complementary to an mRNA sequence. Accordingly, an antisense polynucleotide can bind via hydrogen bonds to a sense polynucleotide. The antisense polynucleotide can be complementary to an entire coding strand of a gene of the invention or to only a portion thereof. In one embodiment, an antisense polynucleotide is antisense to a “coding region” of the coding strand of a nucleotide sequence of the invention. The term “coding region” includes the region of the nucleotide sequence comprising codons which are translated into amino acid. In another embodiment, the antisense polynucleotide is antisense to a “noncoding region” of the coding strand of a nucleotide sequence of the invention.

Antisense polynucleotides of the invention can be designed according to the rules of Watson and Crick base pairing. The antisense polynucleotide can be complementary to the entire coding region of an mRNA corresponding to a gene of the invention, but more preferably is an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense polynucleotide of the invention can be constructed using chemical synthesis and enzymatic ligation reactions using procedures known in the art. For example, an antisense polynucleotide (e.g., an antisense oligonucleotide) can be chemically synthesized using naturally occurring nucleotides or variously modified nucleotides designed to increase the biological stability of the molecules or to increase the physical stability of the duplex formed between the antisense and sense polynucleotides, e.g., phosphorothioate derivatives and acridine substituted nucleotides can be used. Examples of modified nucleotides which can be used to generate the antisense polynucleotide include 5-fluorouracil, 5-bromouracil, 5-chlorouracil, 5-iodouracil, hypoxanthine, xantine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladen4exine, unacil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense polynucleotide can be produced biologically using an expression vector into which a polynucleotide has been subcloned in an antisense orientation (i.e., RNA transcribed from the inserted polynucleotide will be of an antisense orientation to a target polynucleotide of interest, described further in the following subsection).

The antisense polynucleotides of the invention are typically administered to a subject or applied in situ such that they hybridize with or bind to cellular mRNA and/or genomic DNA encoding a MDK gene to thereby inhibit expression of the protein, e.g., by inhibiting transcription and/or translation. The hybridization can be by conventional nucleotide complementarity to form a stable duplex, or, for example, in the cases of an antisense polynucleotide which binds to DNA duplexes, through specific interactions in the major groove of the double helix. An example of a route of administration of antisense polynucleotides of the invention is direct injection at a tissue site (e.g., intestine or blood). Alternatively, antisense polynucleotides can be modified to target selected cells and then administered systemically. For example, for systemic administration, antisense molecules can be modified such that they specifically bind to receptors or antigens expressed on a selected cell surface, e.g., by linking the antisense polynucleotides to peptides or antibodies which bind to cell surface receptors or antigens. The antisense polynucleotides can also be delivered to cells using the vectors described herein. To achieve sufficient intracellular concentrations of the antisense molecules, vector constructs in which the antisense polynucleotides is placed under the control of a strong pol II or pol III promoter are preferred.

In yet another embodiment, the antisense polynucleotide of the invention is an α-anomeric polynucleotide. An α-anomeric polynucleotide forms specific double-stranded hybrids with complementary RNA in which, contrary to the usual β-units, the strands run parallel to each other. The antisense polynucleotide can also comprise a 2′-o-methylribonucleotide or a chimeric RNA-DNA analogue.

In still another embodiment, an antisense polynucleotide is a ribozyme. Ribozymes are catalytic RNA molecules with ribonuclease activity which are capable of cleaving a single-stranded polynucleotide, such as an mRNA, to which they have a complementary region. Thus, ribozymes can be used to catalytically cleave mRNA transcripts of MDK to thereby inhibit translation of said mRNA. A ribozyme having specificity for a MDK polynucleotide can be designed based upon the nucleotide sequence of a MDK gene. An mRNA transcribed from a MDK gene can be used to select a catalytic RNA having a specific ribonuclease activity from a pool of RNA molecules.

Alternatively, expression of a MDK gene can be inhibited by targeting nucleotide sequences complementary to the regulatory region of these genes (e.g., the promoter and/or enhancers) to form triple helical structures that prevent transcription of the gene in target cells.

Expression of MDK gene can also be inhibited using RNA interference (“RNAi”). RNAi is a phenomenon in which the introduction of double-stranded RNA (dsRNA) into certain organisms or cell types causes degradation of the homologous mRNA. First discovered in the nematode Caenorhabditis elegans, RNAi has since been found to operate in a wide range of organisms. For example, in mammalian cells, introduction of long dsRNA (>30 nucleotides) can initiate a potent antiviral response, exemplified by nonspecific inhibition of protein synthesis and RNA degradation. RNA interference provides a mechanism of gene silencing at the mRNA level. In recent years, RNAi has become an endogenous and potent gene-specific silencing technique that uses double-stranded RNAs (dsRNA) to mark a particular transcript for degradation in vivo. It also offers an efficient and broadly applicable approach for gene knock-out. In addition, RNAi technology can be used for therapeutic purposes. For example, RNAi targeting Fas-mediated apoptosis has been shown to protect mice from fulminant hepatitis. RNAi technology has been disclosed in numerous publications, such as U.S. Pat. Nos. 5,919,619, 6,506,559 and PCT Publication Nos. WO99/14346, WO01/70949, WO01/36646, WO00/63364, WO00/44895, WO01/75164, WO01/92513, WO01/68836 and WO01/29058.

A sequence capable of inhibiting gene expression by RNA interference can be in any length. For instance, the sequence can have at least 10, 15, 20, 25, 30, 35, 40, 45, 50, 100, or more consecutive nucleotides. The sequence can be dsRNA or other any type of polynucleotide, provided that the sequence can form a functional silencing complex to degrade the target mRNA transcript.

In one embodiment, the sequence comprises or consists of a short interfering RNA (siRNA). The siRNA can be dsRNA having 19-25 nucleotides. siRNAs can be produced endogenously by degradation of longer dsRNA molecules by an RNase III-related nuclease called Dicer. siRNAs can also be introduced into a cell exogenously or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA takes place near the middle of the region bound by the siRNA strand. This sequence-specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNA are duplexes of short mixed oligonucleotides which can include, for example, 19 nucleotides with symmetric dinucleotide 3′overhangs. Using synthetic 21 bp siRNA duplexes (e.g., 19 RNA bases followed by a UU or dTdT 3′ overhang), sequence-specific gene silencing can be achieved in mammalian cells. These siRNAs can specifically suppress targeted gene translation in mammalian cells without activation of DNA-dependent protein kinase (PKR) by longer dsRNA, which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (pol III) transcription units. Pol III transcription units are suitable for hairpin siRNA expression, since they deploy a short AT rich transcription termination site that leads to the addition of 2 bp overhangs (e.g., UU) to hairpin siRNAs - a feature that is helpful for siRNA function. The Pol III expression vectors can also be used to create transgenic mice that express siRNA.

In another embodiment, siRNAs can be expressed in a tissue-specific manner. Under this approach, long double-stranded RNAs (dsRNAs) are first expressed from a promoter (such as CMV (pol II)) in the nuclei of selected cell lines or transgenic mice. The long dsRNAs are processed into siRNAs in the nuclei (e.g., by Dicer). The siRNAs exit from the nuclei and mediate gene-specific silencing. A similar approach can be used in conjunction with tissue-specific promoters to create tissue-specific knockdown mice.

Any 3′dinucleotide overhang, such as UU, can be used for siRNA design. In some cases, G residues in the overhang are avoided because of the potential for the siRNA to be cleaved by RNase at single-stranded G residues.

With regard to the siRNA sequence itself, it has been found that siRNAs with 30-50% GC content can be more active than those with a higher G/C content in certain cases. Moreover, since a 4-6 nucleotide poly(T) tract may act as a termination signal for RNA pol III, stretches of ≧4 Ts or As in the target sequence may be avoided in certain cases when designing sequences to be expressed from an RNA pol III promoter. In addition, some regions of mRNA may be either highly structured or bound by regulatory proteins. Thus, it may be helpful to select siRNA target sites at different positions along the length of the gene sequence. Finally, the potential target sites can be compared to the appropriate genome database (human, mouse, rat, etc.). Any target sequences with more than 16-17 contiguous base pairs of homology to other coding sequences may be eliminated from consideration in certain cases.

In one embodiment, siRNA can be designed to have two inverted repeats separated by a short spacer sequence and end with a string of Ts that serve as a transcription termination site. This design produces an RNA transcript that is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desirable results.

The siRNA targets can be selected by scanning an mRNA sequence for AA dinucleotides and recording the 19 nucleotides immediately downstream of the AA. Other methods can also been used to select the siRNA targets. In one example, the selection of the siRNA target sequence is purely empirically determined (see e.g., Sui et al., Proc. Natl. Acad. Sci. USA 99: 5515-5520, 2002), as long as the target sequence starts with GG and does not share significant sequence homology with other genes as analyzed by BLAST search. In another example, a more elaborate method is employed to select the siRNA target sequences. This procedure exploits an observation that any accessible site in endogenous mRNA can be targeted for degradation by synthetic oligodeoxyribonucleotide/RNase H method (Lee et al., Nature Biotechnology 20:500-505, 2002).

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In certain instances, the reversal of the order may cause partial reduction in gene silencing activities.

The length of nucleotide sequence being used as the stem of siRNA expression cassette can range, for instance, from 19 to 29. The loop size can range from 3 to 23 nucleotides. Other lengths and/or loop sizes can also be used.

In yet another embodiment, a 5′ overhang in the hairpin siRNA construct can be used, provided that the hairpin siRNA is functional in gene silencing. In one specific example, the 5′ overhang includes about 6 nucleotide residues.

In still yet another embodiment, the target sequence for RNAi is a 21-mer sequence fragment selected from SEQ ID NO:4. The 5′ end of the target sequence has dinucleotide “NA,” where “N” can be any base and “A” represents adenine. The remaining 19-mer sequence has a GC content of between 35% and 55%. In addition, the remaining 19-mer sequence does not include any four consecutive A or T (i.e., AAAA or TTTT) or seven “GC” in a role. Exemplary RNAi target sequences identified according to the above-described criteria are illustrated in Table 2. The siRNA sequence for each target sequence (the sense strand and the antisense strand), and the 5′ end location of each target sequence in SEQ ID NO:4 (“5 End”) are also indicated in Table 2.

Additional criteria can also be used for RNAi target sequence design. For instance, the GC content of the remaining 19-mer sequence can be limited to between 45% and 55%. Moreover, any 19-mer sequence having three consecutive identical bases (i.e., GGG, CCC, TTT, or AAA) or a palindrome sequence with 5 or more bases is excluded. Furthermore, the remaining 1 9-mer sequence can be selected to have low sequence homology to other human genes. In one specific example, potential target sequences are searched by BLASTN against NCBI's human UniGene cluster sequence database. The human UniGene database contains non-redundant sets of gene-oriented clusters. Each UniGene cluster includes sequences that represent a unique gene. 19-mer sequences producing no hit to other human genes under the BLASTN search can be selected. During the search, the e-value may be set at a stringent value (such as “1”).

TABLE 2
Exemplary RNAi Target Sequences of the MDK
Gene and the Corresponding siRNAs

| | | | |
|---|---|---|---|
| Target Sequence | | siRNA Sense Strand | siRNA Antisense Strand |
| (SEQ ID NO) | 5′End | (SEQ ID NO) | (SEQ ID NO) |

| | | | |
|---|---|---|---|
| AAGAAAGATAAGGTGAAGAAG | 94 | GAAAGAUAAGGUGAAGAAGUU | UUCUUUCUAUUCCACUUCUUC |
| (SEQ ID NO:5) | | (SEQ ID NO:6) | (SEQ ID NO:7) |
| AACTGGAAGAAGGAGTTTGGA | 247 | CUGGAAGAAGGAGUUUGGAUU | UUGACCUUCUUCCUCAAACCU |
| (SEQ ID NO:8) | | (SEQ ID NO:9) | (SEQ ID NO:10) |
| AAGTACAAGTTTGAGAACTGG | 277 | GUACAAGUUUGAGAACUGGUU | UUCAUGUUCAAACUCUUGACC |
| (SEQ ID NO:11) | | (SEQ ID NO:12) | (SEQ ID NO:13) |
| AAGACCAAAGCAAAGGCCAAA | 409 | GACCAAAGCAAAGGCCAAAUU | UUCUGGUUUCGUUUCCGGUUU |
| (SEQ ID NO:14) | | (SEQ ID NO:15) | (SEQ ID NO:16) |
| AAAGCAAAGGCCAAAGCCAAG | 415 | AGCAAAGGCCAAAGCCAAGUU | UUUCGUUUCCGGUUUCGGUUC |
| (SEQ ID NO:17) | | (SEQ ID NO:18) | (SEQ ID NO:19) |
| AAGCAAAGGCCAAAGCCAAGA | 416 | GCAAAGGCCAAAGCCAAGAUU | UUCGUUUCCGGUUUCGGUUCU |
| (SEQ ID NO:20) | | (SEQ ID NO:21) | (SEQ ID NO:22) |
| AAAGGCCAAAGCCAAGAAAGG | 420 | AGGCCAAAGCCAAGAAAGGUU | UUUCCGGUUUCGGUUCUUUCC |
| (SEQ ID NO:23) | | (SEQ ID NO:24) | (SEQ ID NO:25) |
| CAACTGGAAGAAGGAGTTTGG | 246 | ACUGGAAGAAGGAGUUUGGUU | UUUGACCUUCUUCCUCAAACC |
| (SEQ ID NO:26) | | (SEQ ID NO:27) | (SEQ ID NO:28) |
| CAAGTACAAGTTTGAGAACTG | 276 | AGUACAAGUUUGAGAACUGUU | UUUCAUGUUCAAACUCUUGAC |
| (SEQ ID NO:29) | | (SEQ ID NO:30) | (SEQ IDNO:31) |
| CAAGACCAAAGCAAAGGCCAA | 408 | AGACCAAAGCAAAGGCCAAUU | UUUCUGGUUUCGUUUCCGGUU |
| (SEQ ID NO:32) | | (SEQ ID NO:33) | (SEQ ID NO:34) |
| CAAAGCAAAGGCCAAAGCCAA | 414 | AAGCAAAGGCCAAAGCCAAUU | UUUUCGUUUCCGGUUUCGGUU |
| (SEQ ID NO:35) | | (SEQ ID NO:36) | (SEQ ID NO:37) |
| CAAAGGCCAAAGCCAAGAAAG | 419 | AAGGCCAAAGCCAAGAAAGUU | UUUUCCGGUUUCGGUUCUUUC |
| (SEQ ID NO:38) | | (SEQ ID NO:39) | (SEQ ID NO:40) |
| GAAGAAGGAGTTTGGAGCCGA | 252 | AGAAGGAGUUUGGAGCCGAUU | UUUCUUCCUCAAACCUCGGCU |
| (SEQ ID NO:41) | | (SEQ ID NO:42) | (SEQ ID NO:43) |
| GAGCCGACTGCAAGTACAAGT | 266 | GCCGACUGCAAGUACAAGUUU | UUCGGCUGACGUUCAUGUUCA |
| (SEQ ID NO:44) | | (SEQ ID NO:45) | (SEQ ID NO:46) |
| GACTGCAAGTACAAGTTTGAG | 271 | CUGCAAGUACAAGUUUGAGUU | UUGACGUUCAUGUUCAAACUC |
| (SEQ ID NO:47) | | (SEQ ID NO:48) | (SEQ ID NO:49) |
| GACCAAAGCAAAGGCCAAAGC | 411 | CCAAAGCAAAGGCCAAAGCUU | UUGGUUUCGUUUCCGGUUUCG |
| (SEQ ID NO:50) | | (SEQ ID NO:51) | (SEQ ID NO:52) |

In yet another embodiment, the polynucleotides of the present invention can be modified at the base moiety, sugar moiety or phosphate backbone to improve, e.g., the stability, hybridization, or solubility of the molecules. For instance, the deoxyribose phosphate backbone of the polynucleotide can be modified to generate peptide polynucleotides. As used herein, the terms “peptide polynucleotides” or “PNAs” refer to polynucleotide mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. PNA oligomers can be synthesized using standard solid phase peptide synthesis protocols.

PNAs can be used in therapeutic and diagnostic applications. For example, PNAs can be used as antisense or antigene agents for sequence-specific modulation of MDK gene expression by, for example, inducing transcription or translation arrest or inhibiting replication. PNAs can also be used in the analysis of single base pair mutations in a gene, (e.g., by PNA-directed PCR clamping); as artificial restriction enzymes when used in combination with other enzymes (e.g., S1 nucleases) or as probes or primers for DNA sequencing or hybridization.

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the polynucleotides of the invention can be generated which may combine the advantageous properties of PNA and DNA. These chimeras allow DNA recognition enzymes, (e.g., RNAse H and DNA polymerases), to interact with the DNA portion while the PNA portion provides high binding affinity and specificity. PNA-DNA chimeras can be linked using linkers of appropriate lengths that are selected based on of base stacking, number of bonds between the nucleobases, and orientation. The synthesis of PNA-DNA chimeras can be performed. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a spacer between the PNA and the 5′ end of DNA. PNA monomers are then coupled in a stepwise manner to produce a chimeric molecule with a 5′ PNA segment and a 3′ DNA segment. Alternatively, chimeric molecules can be synthesized with a 5′ DNA segment and a 3′ PNA segment.

In other embodiments, the oligonucleotide may include other appended groups such as peptides (e.g., for targeting host cell receptors in vivo), or agents facilitating transport across the cell membrane or the blood-kidney barrier. In addition, oligonucleotides can be modified using hybridization-triggered cleavage agents or intercalating agents. To this end, the oligonucleotide may be conjugated to another compound (e.g., a peptide, hybridization triggered cross-linking agent, transport agent, or hybridization-triggered cleavage agent). Finally, the oligonucleotide may be detectably labeled, either such that the label is detected by the addition of another reagent (e.g., a substrate for an enzymatic label), or is detectable immediately upon hybridization of the nucleotide (e.g., a radioactive label or a fluorescent label).

Polypeptides and Variants Thereof

Several aspects of the invention pertain to isolated MDK polypeptides and mutated MDK polypeptides capable of inhibiting normal MDK activity. The present invention also contemplates immunogenic polypeptide fragements suitable for raising anti-MDK antibodies.

In one embodiment, native MDK polypeptides can be isolated from cells or tissue sources by an appropriate purification scheme using standard protein purification techniques. Standard purification methods include electrophoretic, molecular, immunological and chromatographic techniques, including ion exchange, hydrophobic, affinity, and reverse-phase HPLC chromatography, and chromatofocusing. For example, the MDK polypeptides may be purified using a standard anti-MDK antibody column. Ultrafiltration and diafiltration techniques can also be used. The degree of purification necessary depends on the purpose of the MDK polypeptides. In some instances purification will not be necessary.

In another embodiment, MDK polypeptides or mutated MDK polypeptides capable of inhibiting normal MDK activity (dominant-negative mutants) are produced by recombinant DNA techniques. Alternative to recombinant expression, MDK polypeptides or mutated MDK polypeptides can be synthesized chemically using standard peptide synthesis techniques.

The invention provides MDK polypeptides encoded by the human MDK gene, or homologs thereof. In other embodiments, the MDK polypeptide is substantially homologous to a MDK polypeptide encoded by the human MDK gene, and retains the functional activity of the MDK polypeptide yet differs in amino acid sequence due to natural allelic variation or mutagenesis, as described in detail above. Accordingly, in another embodiment, the MDK polypeptide is a protein which comprises an amino acid sequence at least about 60%, 65%, 70%, 75%, 80%, 85%, 90%, 95%, 98% or more homologous to the amino acid sequence encoded by the human MDK gene.

Comparison of sequences and determination of percent homology between two sequences can be accomplished using a mathematical algorithm. In a preferred embodiment, the percent identity between two amino acid sequences is determined using the Needleman and Wunsch (J. Mol. Biol. 48:444-453, 1970) algorithm which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. The percent homology between two amino acid or nucleotide sequences can also be determined using the algorithm of E. Meyers and W. Miller (CABIOS, 4:11-17, 1989) which has been incorporated into the ALIGN program (version 2.0), or the pairwise BLAST program available at NCBI's BLAST web site.

The polypeptide and polynucleotide sequences of the present invention can be used as query sequences for searching public databases in order to identify other family members or related sequences. Such searches can be performed using the NBLAST and XBLAST programs (version 2.0). BLAST nucleotide searches can be performed with the NBLAST program, score=100, wordlength=12 to obtain nucleotide sequences homologous to polynucleotides of the invention. BLAST protein searches can be performed with the XBLAST program, score=50, wordlength=3 to obtain amino acid sequences homologous to MDK. To obtain gapped alignments for comparison purposes, Gapped BLAST can be utilized as described in Altschul et al., (1997) Polynucleotides Res. 25(17):3389-3402. When utilizing BLAST and Gapped BLAST programs, the default parameters of the respective programs (e.g., XBLAST and NBLAST) can be used.

The invention also provides chimeric or fusion MDK polypeptides. A fusion MDK polypeptide contains a MDK-related polypeptide and a non-MDK polypeptide fused in-frame to each other. The MDK-related polypeptide corresponds to all or a portion of a MDK polypeptide or its variant. In a preferred embodiment, a fusion MDK polypeptide comprises at least one portion of a MDK polypeptide sequence recited in SEQ ID NO:1.

A peptide linker sequence may be employed to separate the MDK-related polypeptide from non-MDK polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the MDK-related polypeptide and non-MDK polypeptide; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences suitable as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequences may generally be from about 1 to about 50 amino acids in length. Linker sequences are not required when the MDK-related polypeptide or the non-MDK polypeptide have non-essential N-terminal amino acid regions that can be used to separate the respective functional domains and thereby prevent steric interference.

For example, in one embodiment, the fusion protein is a GST-MDK fusion protein in which the MDK-related sequences are fused to the C-terminus of the GST sequences. Such fusion proteins can facilitate the purification of recombinant MDKs.

The MDK-fusion proteins of the invention can be incorporated into pharmaceutical compositions and administered to a subject in vivo, as described herein. The MDK-fusion proteins can be used to affect the bioavailability of a MDK substrate. Use of MDK-fusion proteins may be useful therapeutically for the treatment of or prevention of damage caused by, for example, (i) aberrant modification or mutation of MDK, and (ii) aberrant post-translational modification of MDK. It is also conceivable that a fusion protein containing a normal or mutated MDK polypeptide, or a fragment thereof may be capable of inhibiting MDK activity in a subject.

Moreover, the MDK-fusion proteins can be used as immunogens to produce anti-MDK antibodies in a subject, to purify MDK ligands and in screening assays to identify molecules which inhibit the interaction of a MDK with a MDK substrate.

MDK-fusion proteins used as immunogens may comprise a non-MDK immunogenic protein. Preferably the immunogenic protein is capable of eliciting a recall response.

Preferably, MDK-chimeric or fusion proteins of the invention are produced using standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence. Moreover, many expression vectors are commercially available that already encode a fusion moiety (e.g., a GST polypeptide). A MDK-related polynucleotide can be cloned into such an expression vector such that the fusion moiety is linked in-frame to the MDK-related polypeptide

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a polynucleotide sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods.

Alternatively, the signal sequence can be linked to the protein of interest using a sequence which facilitates purification, such as with a GST domain.

The present invention also pertains to variants of MDK which function as antagonists to MDK. In one embodiment, antagonists or agonists of MDK are used as therapeutic agents. For example, antagonists to MDK that can decrease the activity or expression of MDK may ameliorate SLE/LN in a subject wherein MDK is abnormally increased in level or activity. Variants of MDKs can be generated by mutagenesis, e.g., discrete point mutation or truncation of a MDK.

In certain embodiments, an antagonist of a MDK can inhibit one or more of the activities of the naturally occurring form of the MDK by, for example, competitively modulating an activity of the MDK. Thus, specific biological effects can be elicited by treatment with a variant of limited function.

Mutants of a MDK which function as either MDK agonists or as MDK antagonists can be identified by screening combinatorial libraries of mutants. In certain embodiments, such variants may be used for example as a therapeutic protein of the invention. A variegated library of MDK variants can be produced by, for example, enzymatically ligating a mixture of synthetic oligonucleotides into gene sequences such that a degenerate set of potential MDK sequences is expressible as individual polypeptides, or alternatively, as a set of larger fusion proteins (e.g., for phage display) containing the set of MDK sequences therein. There are a variety of methods which can be used to produce libraries of potential MDK variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential MDK sequences. Methods for synthesizing degenerate oligonucleotides are known in the art.

In addition, libraries of fragments of a protein coding sequence corresponding to a MDK can be used to generate a variegated population of MDK fragments for screening and subsequent selection of variants of a MDK. In one embodiment, a library of coding sequence fragments can be generated by treating a double stranded PCR fragment of a MDK coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the MDK.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify MDK variants (Delgrave et al., Protein Engineering 6:327-331, 1993).

Portions of a MDK or variants of a MDK having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. Equipment for automated synthesis of polypeptides is commercially available from suppliers such as Perkin Elmer/Applied BioSystems Division (Foster City, Calif.), and may be operated according to the manufacturer's instructions.

Methods and compositions for screening for protein inhibitors or activators are known in the art (see e.g., U.S. Pat. Nos. 4,980,281, 5,266,464, 5,688,635, and 5,877,007, which are incorporated herein by reference).

Antibodies

In accordance with another aspect of the present invention, antibodies specific to MDK or its variants are prepared. An antibody is considered to bind “specifically” to an antigen if the binding affinity between the antibody and the antigen is equal to or greater than 10⁵ M⁻¹. The antibodies can be monoclonal or polyclonal. Preferably, the antibodies are monoclonal. More preferably, the antibodies are humanized antibodies.

In another aspect, the invention provides methods of making an isolated hybridoma which produces an antibody useful for diagnosing a patient or animal with SLE/LN. In this method, a MDK or its variant is isolated (e.g., by purification from a cell in which it is expressed or by transcription and translation of a polynucleotide encoding the protein in vivo or in vitro using known methods). A vertebrate, preferably a mammal such as a mouse, rabbit or sheep, is immunized using the isolated polypeptide or polypeptide fragment. The vertebrate may optionally (and preferably) be immunized at least one additional time with the isolated polypeptide or polypeptide fragment, so that the vertebrate exhibits a robust immune response to the polypeptide or polypeptide fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the polypeptide or polypeptide fragment. The invention also includes hybridomas made by this method and antibodies made using such hybridomas.

An isolated MDK polypeptide, or a portion or fragment thereof, can be used as an immunogen to generate antibodies that bind the MDK polypeptide using standard techniques for polyclonal and monoclonal antibody preparation. A full-length MDK polypeptide can be used or, alternatively, the invention provides antigenic peptide fragments of the MDK polypeptide for use as immunogens. The antigenic peptide of a MDK polypeptide preferably comprises at least 8 amino acid residues of an amino acid sequence encoded by a MDK gene, and encompasses an epitope of a MDK polypeptide such that an antibody raised against the peptide forms a specific immune complex with the MDK polypeptide. Preferably, the antigenic peptide comprises at least 8 amino acid residues, more preferably at least 12 amino acid residues, even more preferably at least 16 amino acid residues, and most preferably at least 20 amino acid residues.

Immunogenic portions (i.e., epitopes) may generally be identified using well known techniques. Such techniques include screening polypeptides for the ability to react with antigen-specific antibodies, antisera and/or T-cell lines or clones. Such antisera and antibodies may be prepared as described herein, and using well known techniques. An epitope of a MDK polypeptide is a portion that reacts with such antisera and/or T-cells at a level that is not substantially less than the reactivity of the full length polypeptide (e.g., in an ELISA and/or T-cell reactivity assay). Such epitopes may react within such assays at a level that is similar to or greater than the reactivity of the full length polypeptide. Such screens may generally be performed using methods well known to those of ordinary skill in the art. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

Preferred epitopes encompassed by the antigenic peptide are regions of a MDK polypeptide that are located on the surface of the polypeptide, e.g., hydrophilic regions, as well as regions with high antigenicity.

A MDK immunogen typically is used to prepare antibodies by immunizing a suitable subject, (e.g., rabbit, goat, mouse or other mammal) with the immunogen. An appropriate immunogenic preparation can contain, for example, recombinantly expressed MDK or a chemically synthesized MDK. The preparation can further include an adjuvant, such as Freund's complete or incomplete adjuvant, or similar immunostimulatory agent. Immunization of a suitable subject with an immunogenic MDK preparation induces a polyclonal anti-MDK antibody response. Techniques for preparing, isolating and using antibodies are well known in the art.

Accordingly, another aspect of the invention pertains to monoclonal or polyclonal anti-MDK antibodies. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to MDK.

Polyclonal anti-MDK antibodies can be prepared as described above by immunizing a suitable subject with MDK. The anti-MDK antibody titer in the immunized subject can be monitored over time by standard techniques, such as with an enzyme linked immunosorbent assay (ELISA) using immobilized MDK or a fragment of MDK. If desired, the antibody molecules directed against MDK can be isolated from the mammal (e.g., from the blood) and further purified by well known techniques, such as protein A chromatography, to obtain the IgG fraction. At an appropriate time after immunization, e.g., when the anti-MDK antibody titers are the highest, antibody-producing cells can be obtained from the subject and used to prepare monoclonal antibodies by standard techniques, such as the hybridoma technique, human B cell hybridoma technique, the EBV-hybridoma technique, or trioma techniques. The technology for producing monoclonal antibody hybridomas is well known. Briefly, an immortal cell line (typically a myeloma) is fused to lymphocytes (typically splenocytes) from a mammal immunized with a MDK immunogen as described above, and the culture supernatants of the resulting hybridoma cells are screened to identify a hybridoma producing a monoclonal antibody that binds to MDK.

Any of the many well known protocols used for fusing lymphocytes and immortalized cell lines can be applied for the purpose of generating an anti-MDK monoclonal antibody. Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp210-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody are detected by screening the hybridoma culture supernatants for antibodies that bind to MDK specifically, e.g., using a standard ELISA assay.

Alternative to preparing monoclonal antibody-secreting hybridomas, a monoclonal anti-MDK antibody can be identified and isolated by screening a recombinant combinatorial immunoglobulin library (e.g., an antibody phase display library) with MDK to thereby isolate immunoglobulin library members that bind to MDK. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612).

The anti-MDK antibodies also include “Single-chain Fv” or “scFv” antibody fragments. The scFv fragments comprise the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding.

Additionally, recombinant anti-MDK antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

Humanized antibodies are particularly desirable for therapeutic treatment of human subjects. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies), which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the constant regions being those of a human immunoglobulin consensus sequence. The humanized antibody will preferably also comprise at least a portion of an immunoglobulin constant region (Fc), typically that of a human immunoglobulin.

Such humanized antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of a MDK polypeptide. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies.

Humanized antibodies which recognize a selected epitope can be generated using a technique referred to as “guided selection.” In this approach a selected non-human monoclonal antibody, e.g., a murine antibody, is used to guide the selection of a humanized antibody recognizing the same epitope.

In a preferred embodiment, the antibodies to MDK are capable of reducing or eliminating the biological function of MDK. Generally, at least a 25% decrease in activity is preferred, with at least about 50% being particularly preferred and about a 95-100% decrease being especially preferred.

An anti-MDK antibody can be used to isolate MDK by standard techniques, such as affinity chromatography or immunoprecipitation. An anti-MDK antibody can facilitate the purification of natural MDKs from cells and of recombinantly produced MDKs expressed in host cells. Moreover, an anti-MDK antibody can be used to detect MDK (e.g., in a cellular lysate or cell supernatant on the cell surface) in order to evaluate the abundance and pattern of expression of MDK. Anti-MDK antibodies can be used diagnostically to monitor protein levels in tissue as part of a clinical testing procedure, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidinibiotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive materials include ¹²⁵I, ¹³¹I, ³⁵S or ³H.

Anti-MDK antibodies of the invention are also useful for targeting a therapeutic agent/drug to a particular cell or tissue comprising the antigen of the anti-MDK antibody. For example, a therapeutic agent such as a small molecule can be linked to the anti-MDK antibody in order to target the therapeutic to the cell or tissue comprising the MDK antigen.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology. See e.g., U.S. Pat. No. 4,671,958.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and acid-catalyzed hydrolysis (e.g., U.S. Pat. No. 4,569,789).

It may also be desirable to couple more than one agent to an antibody. In one embodiment, agents are coupled to one antibody molecule. In another embodiment, at least two different types of agent may be coupled to one antibody. Regardless of the particular embodiment, immunoconjugates coupled with more than one agent can be prepared in a variety of ways, as appreciated by one or ordinary skill in the art.

Vectors, Expression Vectors and Gene Delivery Vectors

Another aspect of the invention pertains to vectors containing a polynucleotide encoding MDK or a portion thereof. One type of vector is a “plasmid,” which includes a circular double stranded DNA loop into which additional DNA segments can be ligated. In the present specification, “plasmid” and “vector” can be used interchangeably as the plasmid is the most commonly used form of vector. However, the invention is intended to include such other forms of vectors, such as expression vectors, and gene delivery vectors.

The expression vectors of the invention comprise a polynucleotide encoding MDK or a portion thereof in a form suitable for expression of the polynucleotide in a host cell, which means that the expression vectors include one or more regulatory sequences, selected on the basis of the host cells to be used for expression, which is operatively linked to the polynucleotide sequence to be expressed. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by polynucleotides as described herein (e.g., MDK, variants of MDK, MDK fusion proteins, and the like).

The expression vectors of the invention can be designed for expression of MDK or its variants in prokaryotic or eukaryotic cells. For example, MDK can be expressed in bacterial cells such as E. coli, insect cells (e.g., using baculovirus expression vectors) yeast cells or mammalian cells. In certain embodiments, such protein may be used, for example, as a therapeutic protein of the invention. Alternatively, the expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of the recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include PGEX (Pharmacia Piscataway, N.J.), pMAL (New England Biolabs, Beverly, Mass.) and pRITS (Pharmacia, Piscataway, N.J.) which fuse glutathione S transferase (GST), maltose E binding protein, or protein A, respectively, to the target recombinant protein.

Purified fusion proteins can be utilized in MDK activity assays, (e.g., direct assays or competitive assays described in detail below), or to generate antibodies specific for MDK, for example.

Examples of suitable inducible non-fusion E. coli expression vectors include pTrc and pET 11d. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

One strategy to maximize recombinant protein expression in E. coli is to express the protein in host bacteria that have an impaired capacity to proteolytically cleave the recombinant protein. Another strategy is to alter the polynucleotide sequence of the polynucleotide to be inserted into an expression vector so that the individual codons for each amino acid are those preferentially utilized in E. coli. Such alteration of polynucleotide sequences of the invention can be carried out by standard DNA synthesis techniques.

In another embodiment, the MDK expression vector is a yeast expression vector. Examples of vectors for expression in yeast S. cerevisiae include pYepSec 1, pMFa, pJRY88, pYES2 (Invitrogen Corporation, San Diego, Calif.), and picZ (Invitrogen Corp, San Diego, Calif.).

Alternatively, MDK can be expressed in insect cells using baculovirus expression vectors. Suitable baculovirus vectors available for expression of proteins in cultured insect cells (e.g., Sf9 cells) include the pAc series and the pV_{L} series.

In yet another embodiment, MDK or its variant is expressed in mammalian cells using a mammalian expression vector. Examples of mammalian expression vectors include pCDM8 and pMT2PC. When used in mammalian cells, the expression vector's control functions are often provided by viral regulatory elements. For example, commonly used promoters are derived from polyoma, adenovirus 2, cytomegalovirus and Simian Virus 40. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1 ). This viral polymerase is supplied by host strains BL21 (DE3) or HSLE174(DE3) from a resident prophage harboring a T7 gn1 gene under the transcriptional control of the lacUV 5 promoter.

In another embodiment, the mammalian expression vector is capable of directing expression of the polynucleotide preferentially in a particular cell type (e.g., tissue-specific regulatory elements are used to express the polynucleotide). Tissue-specific regulatory elements are known in the art and may include epithelial cell-specific promoters. Examples of suitable tissue-specific promoters include the liver-specific albumin promoter, lymphoid-specific promoters, promoters of T cell receptors and immunoglobulins, neuron-specific promoters (e.g., the neurofilament promoter), pancreas-specific promoters, and mammary gland-specific promoters (e.g., milk whey promoter). Developmentally-regulated promoters are also encompassed, for example the α-fetoprotein promoter.

The present invention also provides a recombinant expression vector comprising a polynucleotide which encodes MDK but is cloned into the expression vector in an antisense orientation. That is, the DNA molecule is operatively linked to a regulatory sequence in a manner which allows for expression (e.g., via transcription of the DNA molecule) of an RNA molecule which is antisense to mRNA corresponding to MDK gene. Regulatory sequences operatively linked to a polynucleotide cloned in the antisense orientation can be chosen to direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense polynucleotides are produced under the control of a higly efficient regulatory region, the activity of which can be determined by the cell type into which the vector is introduced.

The present invention further provides gene delivery vehicles for delivery of polynucleotides to cells, tissue, or a mammal for expression. For example, a polynucleotide sequence of the invention can be administered either locally or systemically in a gene delivery vehicle. These constructs can utilize viral or non-viral vector approaches in in vivo or ex vivo modality. Expression of such coding sequence can be induced using endogenous mammalian or heterologous promoters. Expression of the coding sequence in vivo can be either constituted or regulated. The invention includes gene delivery vehicles capable of expressing the contemplated polynucleotides. The gene delivery vehicle is preferably a viral vector and, more preferably, a retroviral, lentiviral, adenoviral, adeno-associated viral (AAV), herpes viral, or alphavirus vectors. The viral vector can also be an astrovirus, coronavirus, orthomyxovirus, papovavirus, paramyxovirus, parvovirus, picomavirus, poxvirus, or togavirus viral vector.

Delivery of the gene therapy constructs of this invention into cells is not limited to the above mentioned viral vectors. Other delivery methods and media may be employed such as, for example, nucleic acid expression vectors, polycationic condensed DNA linked or unlinked to killed adenovirus alone, liposomes, ligand linked DNA, eucaryotic cell delivery vehicles, deposition of photopolymerized hydrogel materials, handheld gene transfer particle gun, ionizing radiation, nucleic charge neutralization or fusion with cell membranes. Particle mediated gene transfer may be employed. For example, the sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose or transferrin. Naked DNA may also be employed. The uptake efficiency of the naked DNA may be improved using biodegradable latex beads. DNA coated latex beads are efficiently transported into cells after endocytosis initiation by the beads. This method may be improved further by treatment of the beads to increase hydrophobicity and thereby facilitate disruption of the endosome and release of the DNA into the cytoplasm.

Regulatable Expression Systems

Another aspect of the invention pertains to the expression of polynucleotides or polypeptides that are capable of inhibiting MDK activity or MDK expression using a regulatable expression system. Systems to regulate expression of therapeutic genes have been developed and incorporated into the current viral and nonviral gene delivery vectors. These systems are briefly described below:

Tet-on/off system. The Tet-system is based on two regulatory elements derived from the tetracycline-resistance operon of the E. coli Tn10 transposon: the tet repressor protein (TetR) and the Tet operator DNA sequence (tetO) to which TetR binds. The system consists of two components, a “regulator” and a “reporter” plasmid. The “regulator” plasmid encodes a hybrid protein containing a mutated Tet repressor (rtetR) fused to the VP16 activation domain of herpes simplex virus. The “reporter” plasmid contains a tet-responsive element (TRE), which controls the “reporter” gene of choice. The rtetR-VP16 fusion protein can only bind to the TRE, therefore activating the transcription of the “reporter” gene in the presence of tetracycline. The system has been incorporated into a number of viral vectors including retrovirus, adenovirus and AAV (Gossen et al., Science 268: 1766-1769, 1995).

Ecdysone system. The Ecdysone system is based on the molting induction system found in Drosophila, but modified for inducible expression in mammalian cells. The system uses an analog of the Drosophila steroid hormone ecdysone, muristerone A, to activate expression of the gene of interest via a heterodimeric nuclear receptor. Expression levels have been reported to exceed 200-fold over basal levels with no effect on mammalian cell physiology (No et al., Proc. Natl. Acad. Sci. USA 93: 3346-3351, 1996).

Progesterone-system. The progesterone receptor is normally stimulated to bind to a specific DNA sequence and to activate transcription through an interaction with its hormone ligand. Conversely, the progesterone antagonist mifepristone (RU486) is able to block hormone-induced nuclear transport and subsequent DNA binding. A mutant form of the progesterone receptor that can be stimulated to bind through an interaction with RU486 has been generated. To generate a specific, regulatable transcription factor, the RU486-binding domain of the progesterone receptor has been fused to the DNA-binding domain of the yeast transcription factor GAL4 and the transactivation domain of the HSV protein VP16. The chimeric factor is inactive in the absence of RU486. The addition of hormone, however, induces a conformational change in the chimeric protein, and this change allows binding to a GAL4-binding site and the activation of transcription from promoters containing the GAL4-binding site (Wang et al., Nat. Biotech 15: 239-243, 1997).

Rapamycin-system. Immunosuppressive agents, such as FK506 and rapamycin, act by binding to specific cellular proteins and facilitating their dimerization. For example, the binding of rapamycin to FK506-binding protein (FKBP) results in its heterodimerization with another rapamycin binding protein FRAP, which can be reversed by removal of the drug. The ability to bring two proteins together by addition of a drug potentiates the regulation of a number of biological processes, including transcription. A chimeric DNA-binding domain has been fused to the FKBP, which enables binding of the fusion protein to a specific DNA-binding sequence. A transcriptional activation domain also has been fused to FRAP. When these two fusion proteins are co-expressed in the same cell, a fully functional transcription factor can be formed by heterodimerization mediated by addition of rapamycin. The dimerized chimeric transcription factor can then bind to a synthetic promoter sequence containing copies of the synthetic DNA-binding sequence. This system has been successfully integrated into adenoviral and AAV vectors. Long term regulatable gene expression has been achieved in both mice and baboons (Ye et al., Science 283: 88-91, 1999).

Detection Methods

As discussed earlier, the expression level of MDK may be used as a marker for SLE/LN. Detection and measurement of the relative amount of a MDK gene product can be carried out using various methods known in the art.

Typical methodologies for detection of a transcribed polynucleotide include RNA extraction from a cell or tissue sample, followed by hybridization of a labeled probe (i.e., a complementary polynucleotide) specific for the target RNA to the extracted RNA and detection of the probe (i.e., Northern blotting).

Typical methodologies for peptide detection include protein extraction from a cell or tissue sample, followed by binding of an antibody specific for the target protein to the protein sample, and detection of the antibody. For example, detection of midkine may be accomplished using polyclonal anti-midkine antibody. Antibodies are generally detected by the use of a labeled secondary antibody. The label can be a radioisotope, a fluorescent compound, an enzyme, an enzyme co-factor, or ligand. Such methods are well understood in the art.

In certain embodiments, the MDK gene itself (i.e., the DNA or cDNA) may serve as a marker for SLE/LN. For example, an increase of genomic copies of a MDK gene, such as by duplication of the gene, may be correlated with SLE/LN.

Detection of specific polynucleotides may also be assessed by gel electrophoresis, column chromatography, or direct sequencing, quantitative PCR (in the case of polynucleotide), RT-PCR, or nested-PCR among many other techniques well known to those skilled in the art.

Detection of the presence or number of copies of all or a part of a MDK gene may be performed using any method known in the art. Typically, it is convenient to assess the presence and/or quantity of a DNA or cDNA by Southern analysis, in which total DNA from a cell or tissue sample is extracted, is hybridized with a labeled probe (i.e., a complementary DNA molecules), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Screening Methods

The present invention also provides methods (also referred to herein as “screening assays”) for identifying modulators, i.e., candidate or test compounds or agents comprising therapeutic moieties (e.g., peptides, peptidomimetics, peptoids, polynucleotides, small molecules or other drugs) which (a) bind to MDK, or (b) have a modulatory (e.g., stimulatory or inhibitory) effect on the activity of MDK or, more specifically, (c) have a modulatory effect on the interactions of MDK with one or more of its natural substrates (e.g., peptide, protein, hormone, co-factor, or polynucleotide), or (d) have a modulatory effect on the expression of MDK. Such assays typically comprise a reaction between MDK and one or more assay components. The other components may be either the test compound itself, or a combination of test compound and a binding partner of MDK.

The test compounds of the present invention are generally inorganic molecules, small organic molecules, and biomolecules. Biomolecules include, but are not limited to, polypeptides, polynucleotides, polysaccharides, as well as any naturally-occurring or synthetic organic compounds that have a bioactivity in mammals. In one preferred embodiment, the test compound is a small organic molecule. In another preferred embodiment, the test compound is a biomolecule.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (e.g., libraries of molecules having the functionalities of peptides, but with a novel, non-peptide backbone which are resistant to enzymatic degradation but which nevertheless remain bioactive; see e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997).

The present invention further includes a method for screening compounds capable of modulating the binding between MDK and a binding partner. As used herein, the term “binding partner” refers to a bioactive agent which serves as either a substrate for MDK, or a ligand having a binding affinity to MDK. The bioactive agent may be selected from a variety of naturally-occurring or synthetic compounds, proteins, peptides, polysaccharides, nucleotides or polynucleotides.

Screening for Inhibitors of MDK

The present invention provides methods of screening test compounds for inhibitors of MDK, and to the pharmaceutical compositions comprising the test compounds. The method of screening comprises obtaining samples from subjects diagnosed with or suspected of having SLE/LN, contacting each separate aliquot of the samples with one of a plurality of test compounds, and comparing expression of MDK in each of the aliquots to determine whether any of the test compounds provides a substantially decreased level of expression or activity of MDK relative to samples with other test compounds or relative to an untreated sample or control sample. In addition, methods of screening may be devised by combining a test compound with a protein and thereby determining the effect of the test compound on the protein.

In addition, the invention is further directed to a method of screening for test compounds capable of modulating with the binding of MDK and a binding partner, by combining the test compound, MDK, and binding partner together and determining whether binding of the binding partner and MDK occurs. The test compound may be either small molecules or a bioactive agent. As discussed below, test compounds may be provided from a variety of libraries well known in the art.

Inhibitors of MDK expression, activity or binding ability are useful as therapeutic compositions of the invention. Such inhibitors may be formulated as pharmaceutical compositions, as described herein below. Such modulators may also be used in the methods of the invention, for example, to diagnose, treat, or prognose SLE/LN.

High-Throughput Screening Assays

The present invention also provides methods for conducting high-throughput screening for test compounds capable of inhibiting activity or expression of MDK. In one embodiment, the high-throughput screening method involves contacting test compounds with MDK and then detecting the effect of the test compounds on MDK. Functional assays such as cytosensor microphysiometer-based assays, calcium flux assays such as FLIPR® (Molecular Devices Corp, Sunnyvale, Calif.), or the TUNEL assay may be employed to measure cellular activity, as discussed below.

A variety of high-throughput functional assays well-known in the art may be used in combination to screen and/or study the reactivity of different types of activating test compounds. Since the coupling system is often difficult to predict, a number of assays may need to be configured to detect a wide range of coupling mechanisms. Fluoroescence-based techniques are well-known in the art and are capable of high-throughput and ultra high throughput screening. They include, but are not limited to BRET® and FRET® (both by Packard Instrument Co., Meriden, Conn.). The ability to screen a large volume and a variety of test compounds with great sensitivity permits for analysis of the therapeutic targets of the invention to further provide potential inhibitors of SLE/LN. The BIACORE® system may also be manipulated to detect binding of test compounds with individual components of the therapeutic target, to detect binding to either the encoded protein or to the ligand.

By combining test compounds with MDK and determining the binding activity between such, diagnostic analysis can be performed to elucidate the coupling systems. Generic assays using cytosensor microphysiometer may also be used to measure metabolic activation, while changes in calcium mobilization can be detected by using the fluorescence-based techniques such as FLIPR® (Molecular Devices Corp, Sunnyvale, Calif.). In addition, the presence of apoptotic cells may be determined by the TUNEL assay, which utilizes flow cytometry to detect free 3-OH termini resulting from cleavage of genomic DNA during apoptosis. As mentioned above, a variety of functional assays well-known in the art may be used in combination to screen and/or study the reactivity of different types of activating test compounds. In a preferred embodiment, the high-throughput screening assay of the present invention uses label-free plasmon resonance technology as provided by BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and polynucleotides), and since the BIACORE® sensor surface can be functionalized to bind a variety of these bioactive agents, detection of a wide selection of test compounds can thus be accomplished.

Therefore, the invention provides for high-throughput screening of test compounds for the ability to inhibit an activity of MDK, by combining the test compounds and MDK in high-throughput assays such as BIACORE®, or in fluorescence-based assays such as BRET®. In addition, high-throughput assays may be utilized to identify specific factors which bind to MDK, or alternatively, to identify test compounds which prevent binding of MDK to the binding partner. Moreover, the high-throughput screening assays may be modified to determine whether test compounds can bind to either MDK or to a binding partner of MDK.

Diagnostic Assays

An exemplary method for detecting the presence of MDK or polynucleotide encoding MDK in a biological sample involves obtaining a biological sample from a test subject and contacting the biological sample with a compound or an agent capable of detecting the protein or polynucleotide (e.g., mRNA, genomic DNA) that encodes MDK such that the presence of MDK or polynucleotide is detected in the biological sample. A preferred agent for detecting mRNA or genomic DNA corresponding to a MDK gene or MDK protein is a labeled polynucleotide probe capable of hybridizing to a MDK mRNA or a genomic DNA. Suitable probes for use in the diagnostic assays of the invention are described herein. A preferred agent for detecting MDK is a MDK-specific antibody which specifically recognizes MDK.

The diagnostic assays may also be used to quantify the amount of expression or activity of MDK in a biological sample. Such quantification is useful, for example, to determine the progression or severity of SLE/LN. Such quantification is also useful, for example, to determine the severity of SLE/LN following treatment.

Determining severity of SLE/LN

In the field of diagnostic assays, the invention also provides methods for determining the severity of SLE/LN by isolating a sample from a subject, detecting the presence, quantity and/or activity of MDK in the sample relative to a second sample from a normal sample or control sample. In one embodiment, the expression levels of MDK in the two samples are compared, and an increased MDK expression in the test sample indicates SLE/LN.

A preferred agent for detecting MDK is an antibody capable of binding to MDK, preferably an antibody with a detectable label. Antibodies can be polyclonal or more preferably, monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect MDK mRNA, protein or genomic DNA in a biological sample in vitro as well as in vivo. For example, in vitro techniques for detection of MDK mRNA include Northern hybridizations and in situ hybridizations. In vitro techniques for detection of MDK include enzyme linked immunosorbent assays (ELISAs), Western blots, immunoprecipitations and immunofluorescence. In vitro techniques for detection of MDK genomic DNA include Southern hybridizations. Furthermore, in vivo techniques for detection of MDK include introducing into a subject a labeled anti-MDK antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject, e.g., a biopsy or blood draw.

Prognostic Assays

The detection methods described herein can furthermore be utilized to identify subjects having or at risk of developing SLE/LN associated with aberrant MDK expression or activity.

Furthermore, the prognostic assays described herein can be used to determine whether a subject can be administered an agent (e.g., an agonist, antagonist, peptidomimetic, protein, peptide, polynucleotide, small molecule, or other drug candidate) to treat or prevent SLE/LN associated with aberrant MDK expression or activity, such as, for example, a cytokine. For example, such methods can be used to determine whether a subject can be effectively treated with an agent to inhibit SLE/LN. Thus, the present invention provides methods for determining whether a subject can be effectively treated with an agent for SLE/LN associated with increased MDK expression or activity in which a test sample is obtained and MDK protein or polynucleotide expression or activity is detected (e.g., wherein the abundance of MDK protein or polynucleotide expression or activity is diagnostic for a subject that can be administered the agent to treat injury associated with aberrant MDK expression or activity).

Prognostic assays can be devised to determine whether a subject undergoing treatment for SLE/LN has a poor outlook for long term survival or disease progression. In a preferred embodiment, prognosis can be determined shortly after diagnosis, i.e., within a few days. By establishing MDK expression profiles of different stages of SLE/LN, from onset to later stages, an expression pattern may emerge to correlate a particular expression profile to increased likelihood of a poor prognosis. The prognosis may then be used to devise a more aggressive treatment program and enhance the likelihood of long-term survival and well being.

The methods of the invention can also be used to detect genetic alterations in a MDK gene, thereby determining if a subject with the altered gene is at risk for damage characterized by aberrant regulation in MDK activity or polynucleotide expression. In preferred embodiments, the methods include detecting, in a sample of cells from the subject, the presence or absence of a genetic alteration characterized by at least one alteration affecting the integrity of a MDK gene, or the aberrant expression of the MDK gene. For example, such genetic alterations can be detected by ascertaining the existence of at least one of the following: 1) deletion of one or more nucleotides from a MDK gene; 2) addition of one or more nucleotides to a MDK gene; 3) substitution of one or more nucleotides of a MDK gene, 4) a chromosomal rearrangement of a MDK gene; 5) alteration in the level of a messenger RNA transcript of a MDK gene, 6) aberrant modification of a MDK gene, such as of the methylation pattern of the genomic DNA, 7) the presence of a non-wild type splicing pattern of a messenger RNA transcript of a MDK gene, 8) non-wild type level MDK, 9) allelic loss of a MDK gene, and 10) inappropriate post-translational modification of MDK. As described herein, there are a large number of assays known in the art, which can be used for detecting alterations in a MDK gene. A preferred biological sample is a blood sample isolated by conventional means from a subject.

In certain embodiments, detection of the alteration involves the use of a probe/primer in a polymerase chain reaction (PCR), such as anchor PCR or RACE PCR, or, alternatively, in a ligation chain reaction (LCR), the latter of which can be particularly useful for detecting point mutations in the MDK gene. This method can include the steps of collecting a sample of cells from a subject, isolating polynucleotide (e.g., genomic, mRNA or both) from the cells of the sample, contacting the polynucleotide sample with one or more primers which specifically hybridize to a MDK gene under conditions such that hybridization and amplification of the MDK gene (if present) occurs, and detecting the presence or absence of an amplification product, or detecting the size of the amplification product and comparing the length to a control sample. It is understood that PCR and/or LCR may be desirable to use as a preliminary amplification step in conjunction with any of the techniques used for detecting mutations described herein.

Alternative amplification methods include: self sustained sequence replication (Guatelli et al., Proc. Natl. Acad. Sci. USA 87:1874-1878, 1990), transcriptional amplification system (Kwoh et al., Proc. Natl. Acad. Sci. USA 86:1173-1177, 1989), Q-Beta Replicase (Lizardi et al., Bio-Technology 6:1197, 1988), or any other polynucleotide amplification method, followed by the detection of the amplified molecules using techniques well known to those of skill in the art. These detection schemes are especially useful for the detection of polynucleotides if such molecules are present in very low numbers.

In an alternative embodiment, mutations in a MDK gene from a sample cell can be identified by alterations in restriction enzyme cleavage patterns. For example, samples and control DNA are isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes can be used to score for the presence of specific mutations by development or loss of a ribozyme cleavage site. See e.g, U.S. Pat. No. 5,498,531.

In other embodiments, genetic mutations in a MDK gene can be identified by hybridizing a sample and control polynucleotides, e.g., DNA or RNA, to high density arrays containing hundreds or thousands of oligonucleotides probes. For example, genetic mutations in a MDK gene can be identified in two dimensional arrays containing light generated DNA probes. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the MDK gene and detect mutations by comparing the sequence of the sample MDK gene with the corresponding wild-type (control) sequence. It is also contemplated that any of a variety of automated sequencing procedures can be utilized when performing the diagnostic assays, including sequencing by mass spectrometry.

Other methods for detecting mutations in a MDK gene include methods in which protection from cleavage agents is used to detect mismatched bases in RNA/RNA or RNA/DNA heteroduplexes (Myers et al., Science 230:1242, 1985). In general, “mismatch cleavage” technique involves forming heteroduplexes by hybridizing a RNA or DNA (labeled) containing the wild-type MDK gene sequence to a potentially mutant RNA or DNA obtained from a tissue sample. The double-stranded duplexes are treated with an agent which cleaves single-stranded regions of the duplex which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in MDK cDNAs obtained from samples of cells. For example, the mutY enzyme of E. coli cleaves A at G/A mismatches and the thymidine DNA glycosylase from HeLa cells cleaves T at G/T mismatches. According to an exemplary embodiment, a probe based on a MDK gene sequence, e.g., a wild-type MDK gene sequence, is hybridized to cDNA or other DNA product from a test cell(s). The duplex thus formed is treated with a DNA mismatch repair enzyme, and the cleavage products, if any, can be detected from electrophoresis protocols or the like. See e.g., U.S. Pat. No. 5,459,039.

In other embodiments, alterations in electrophoretic mobility will be used to identify mutations in a MDK gene. For example, single strand conformation polymorphism (SSCP) may be used to detect differences in electrophoretic mobility between mutant and wild type polynucleotides. Single-stranded DNA fragments of sample and control MDK polynucleotides will be denatured and allowed to renature. The secondary structure of single-stranded polynucleotides varies according to sequence. The resulting alteration in electrophoretic mobility enables the detection of even a single base change. The DNA fragments may be labeled or detected with labeled probes. The sensitivity of the assay may be enhanced by using RNA (rather than DNA) in which the secondary structure is more sensitive to a change in sequence. In a preferred embodiment, the assay utilizes heteroduplex analysis to separate double stranded heteroduplex molecules on the basis of changes in electrophoretic mobility (Keen et al., Trends Genet 7:5, 1991).

In yet another embodiment the movement of mutant or wild-type fragments in polyacrylamide gels containing a gradient of denaturant is assayed using denaturing gradient gel electrophoresis (DGGE). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, Biophys Chem 265:12753, 1987).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al., Proc. Natl. Acad. Sci USA 86:6230, 1989). Such allele specific oligonucleotides are hybridized to PCR amplified target or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) or at the extreme 3′ end of one primer where, under appropriate conditions, mismatch can prevent or reduce polymerase extension. See, for example, Saiki et al., Proc. Natl. Acad. Sci USA 86:6230, 1989) In addition it may be desirable to introduce a novel restriction site in the region of the mutation to create cleavage-based detection. It is anticipated that in certain embodiments amplification may also be performed using Taq ligase for amplification. In such cases, ligation will occur only if there is a perfect match at the 3′ end of the 5′ sequence making it possible to detect the presence of a known mutation at a specific site by looking for the presence or absence of amplification.

The methods described herein may be performed, for example, by using prepackaged diagnostic kits comprising at least one polynucleotide probe or one antibody of the present invention. These kits can be in clinical settings to diagnose subjects exhibiting symptoms or family history of SLE/LN. Furthermore, any cell type or tissue in which MDK is expressed may be used in the prognostic or diagnostic assays described herein.

Monitoring Effects During Clinical Trials

Monitoring the influence of agents (e.g., drugs, small molecules, proteins, nucleotides) on the expression or activity of MDK can be applied not only in basic drug screening, but also in clinical trials. For example, the effectiveness of an agent determined by a screening assay, as described herein to decrease MDK expression, protein levels, or downregulate MDK activity, can be monitored in clinical trials of subjects exhibiting increased MDK expression, protein levels, or upregulated MDK activity. In such clinical trials, the expression or activity of MDK can be used as a “read out” of the phenotype of a particular tissue.

For example, to study the effect of agents on MDK-associated damage in a clinical trial, cells can be isolated and RNA prepared and analyzed for the levels of expression of MDK. The levels of gene expression can be quantified by northern blot analysis, RT-PCR, GeneChip® or Taqman analysis as described herein, or alternatively by measuring the amount of protein produced, by one of the methods as described herein, or by measuring the levels of activity of MDK. In this way, the gene expression level can serve as a read-out, indicative of the physiological response of the cells to the agent. Accordingly, this response state may be determined before treatment and at various points during treatment of the individual with the agent.

In a preferred embodiment, the present invention provides a method for monitoring the effectiveness of treatment of a subject with an agent (e.g., an agonist, antagonist, peptidomimetic, protein, peptide, polynucleotide, small molecule, or other drug candidate identified by the screening assays described herein) including the steps of (i) obtaining a pre-administration sample from a subject prior to administration of the agent; (ii) detecting the level of expression of MDK protein or mRNA in the pre-administration sample; (iii) obtaining one or more post-administration samples from the subject; (iv) detecting the level of expression or activity of MDK protein or mRNA in the post-administration samples; (v) comparing the level of expression or activity of MDK protein or mRNA in the pre-administration sample with the level of expression or activity of MDK protein or mRNA the post administration sample or samples; and (vi) altering the administration of the agent to the subject accordingly. For example, decreased administration of the agent may be desirable to decrease expression or activity of MDK to lower levels than detected, i.e., to decrease the effectiveness of the agent. According to such an embodiment, MDK expression or activity may be used as an indicator of the effectiveness of an agent, even in the absence of an observable phenotypic response.

Methods of Treatment

The present invention provides for both prophylactic and therapeutic methods of treating a subject at risk for, susceptible to or diagnosed with SLE/LN. With regard to both prophylactic and therapeutic methods of treatment, such treatments may be specifically tailored or modified, based on knowledge obtained from the field of pharmacogenomics. “Pharmacogenomics,” as used herein, includes the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype” or “drug response genotype”). Thus, another aspect of the invention provides methods for tailoring an individual's prophylactic or therapeutic treatment with MDK modulators according to that individual's drug response. Pharmacogenomics allows a clinician or physician to target prophylactic or therapeutic treatments to subjects who will most benefit from the treatment and to avoid treatment of subjects who will experience toxic drug-related side effects.

Prophylactic Methods

The present invention further provides a method for preventing in a subject SLE/LN associated with aberrant MDK expression or activity, by administering to the subject an agent which modulates MDK protein expression or activity.

Subjects at risk for SLE/LN which is caused or contributed to by aberrant MDK expression or activity can be identified by, for example, any or a combination of diagnostic or prognostic assays as described herein.

Administration of a prophylactic agent can occur prior to the manifestation of symptoms characteristic of the differential MDK protein expression, such that SLE/LN is prevented or, alternatively, delayed in its progression. Depending on the type of MDK aberrancy (e.g., typically a modulation outside the normal standard deviation), for example, a MDK mutant protein, MDK antagonist agent, or MDK antisense polynucleotide can be used for treating the subject. The appropriate agent can be determined based on screening assays described herein.

Therapeutic Methods

Another aspect of the invention pertains to methods of modulating MDK protein expression or activity for therapeutic purposes. Accordingly, in an exemplary embodiment, the modulatory method of the invention involves contacting a cell with an agent that inhibits MDK gene expression or one or more of the activities of MDK protein associated with the cell. An agent that modulates MDK gene expression or protein activity can be an agent as described herein, such as a polynucleotide, a polypeptide, or a polysaccharide, a naturally-occurring target molecule of a MDK protein (e.g., a MDK protein substrate or receptor), an anti-MDK antibody, a MDK antagonist, a peptidomimetic of a MDK antagonist, or other small organic and inorganic molecule.

These modulatory methods can be performed in vivo (e.g., by administering the agent to a subject). As such, the present invention provides methods of treating an individual diagnosed with or at risk for SLE/LN characterized by aberrant expression or activity of MDK. In one embodiment, the method involves administering an agent (e.g., an agent identified by a screening assay described herein), or combination of agents that downregulates MDK expression or activity. The agent may include a vector comprising a polynucleotide encoding a MDK inhibitor or an antisense sequence. The agent may be an anti-MDK antibody, a plurality of anti-MDK antibodies or an anti-MDK antibody conjugated to a therapeutic moiety. Treatment with the antibody may further be localized to the tissues or cells affected by SLE/LN.

Pharmacogenomics

In conjunction with treatment for SLE/LN using a MDK modulator, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a MDK modulator as well as tailoring the dosage and/or therapeutic regimen of treatment with a MDK modulator.

Pharmacogenomics deals with clinically significant hereditary variations in the response to drugs due to altered drug disposition and abnormal action in affected persons. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is hemolysis after ingestion of oxidant drugs (anti-malarials, sulfonamides, analgesics, nitrofurans) and consumption of fava beans.

One pharmacogenomics approach to identifying genes that predict drug response, known as “a genome-wide association,” relies primarily on a high-resolution map of the human genome consisting of already known gene-related sites (e.g., a “bi-allelic” gene marker map which consists of 60,000-100,000 polymorphic or variable sites on the human genome, each of which has two variants). Such a high-resolution genetic map can be compared to a map of the genome of each of a statistically substantial number of subjects taking part in a Phase II/III drug trial to identify genes associated with a particular observed drug response or side effect. Alternatively, such a high resolution map can be generated from a combination of some ten-million known single nucleotide polymorphisms (SNPs) in the human genome. A “SNP” is a common alteration that occurs in a single nucleotide base in a stretch of DNA. For example, a SNP may occur once per every 1000 bases of DNA. A SNP may be involved in a disease process. However, the vast majority of SNPs may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals. Thus, mapping of the MDK gene to SNP maps of LN patients may allow easier identification of these genes according to the genetic methods described herein.

Alternatively, a method termed the “candidate gene approach,” can be utilized to identify genes that predict drug response. According to this method, if a gene that encodes a drug target is known (e.g., MDK), all common variants of that gene can be fairly easily identified in the population. It then can be determined if a particular drug response is associated with one version of the gene versus another is associated with a particular drug response.

The activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer and poor metabolizer. The prevalence of poor metabolizer phenotypes is different among different populations. For example, the gene coding for CYP2D6 is highly polymorphic and several mutations have been identified in poor metabolizers, which all lead to the absence of functional CYP2D6. Poor metabolizers of CYP2D6 and CYP2C19 quite frequently experience exaggerated drug response and side effects when they receive standard doses. If a metabolite is the active therapeutic moiety, poor metabolizers show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

In one embodiment, a method termed the “gene expression profiling” method can be utilized to identify genes that predict drug response. In this regard, the gene expression profile of an animal dosed with a drug (e.g., MDK expression in response to a MDK modulator) can give an indication of whether the gene pathways related to toxicity have been turned on.

Information generated from more than one of the above pharmacogenomics approaches can be used to determine the appropriate dosage or treatment regimen suitable for a particular individual. This knowledge can avoid adverse reactions or therapeutic failure and thus enhance therapeutic or prophylactic efficiency when treating a subject with a MDK modulator.

Pharmaceutical Compositions

The present invention is further directed to pharmaceutical compositions comprising a MDK modulator and a pharmaceutically acceptable carrier.

As used herein, a “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active modulator (e.g., an anti-MDK antibody, a MDK activity inhibitor, or a gene therapy vector expressing antisense nucleotide to MDK) in the required amount in an appropriate solvent, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Kits

The invention also encompasses kits for detecting the presence of a MDK gene product in a biological sample. An example the kit comprises reagents for assessing expression of MDK at nucleotide or protein level. Preferably, the reagents include an antibody or fragment thereof, wherein the antibody or fragment specifically binds to MDK. For example, antibodies of interest may be prepared by methods known in the art. Optionally, the kits may comprise a polynucleotide probe capable of specifically binding to a transcript of the MDK gene. The kit may also contain means for determining the amount of MDK protein or mRNA in the test sample; and/pr means for comparing the amount of the MDK protein or mRNA in the test sample to a control or standard. The compound or agent can be packaged in a suitable container. The kit can further comprise instructions for using the kit to detect MDK protein or polynucleotide

The invention further provides kits for assessing the suitability of each of a plurality of compounds for inhibiting SLE/LN in a subject. Such kits include a plurality of compounds to be tested, and a reagent (e.g., an antibody specific to corresponding proteins, or a probe or primer specific to corresponding polynucleotides) for assessing expression of MDK.

It should be understood that the above-described embodiments are given by way of illustration, not limitation. Various changes and modifications within the scope of the present invention will become apparent to those skilled in the art from the present description.

Host Cells

Another aspect of the invention pertains to host cells into which a polynucleotide of the invention is introduced, e.g., a MDK gene or homolog thereof, within an expression vector, a gene delivery vector, or a polynucleotide of the invention containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a MDK gene can be expressed in bacterial cells such as E. coli, insect cells, yeast or mammalian cells (e.g., Chinese hamster ovary cells (CHO), COS cells, Fischer 344 rat cells, HLA-B27 rat cells, HeLa cells, A549 cells, or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign polynucleotide (e.g., DNA) into a host cell, including calcium phosphate or calcium chloride co-precipitation, DAKD-dextran-mediated transfection, lipofection, or electoporation.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Preferred selectable flags include those which confer resistance to drugs, such as G418, hygromycin and methotrexate. Polynucleotides encoding a selectable flag can be introduced into a host cell by the same vector as that encoding MDK or can be introduced by a separate vector. Cells stably transfected with the introduced polynucleotide can be identified by drug selection (e.g., cells that have incorporated the selectable flag gene will survive, while the other cells die).

A host cell of the invention, such as a prokaryotic or eukaryotic host cell in culture, can be used to produce (i.e., express) MDK. Accordingly, the invention further provides methods for producing MDK using the host cells of the invention. In one embodiment, the method comprises culturing the host cell of invention (into which a recombinant expression vector containing a MDK gene has been introduced) in a suitable medium such that MDK is produced. In another embodiment, the method further comprises isolating MDK from the medium or the host cell.

Transgenic and Knockout Animals

The host cells of the invention can also be used to produce non-human transgenic animals. For example, in one embodiment, a host cell of the invention is a fertilized oocyte or an embryonic stem cell into which MDK-coding sequences have been introduced. Such host cells can then be used to create non-human transgenic animals in which exogenous sequences encoding MDK have been introduced into their genome or homologous recombinant animals in which endogenous sequences encoding MDK have been altered. Such animals are useful for studying the function and/or activity of MDK and for identifying and/or evaluating modulators of MDK activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” or “knockout animal” is a non-human animal, preferably a mammal, more preferably a mouse, in which an endogenous MDK gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a MDK-encoding polynucleotide into the mate pronuclei of a fertilized oocyte, e.g., by microinjection or retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a transgene to direct expression of MDK to particular cells. Methods for generating transgenic animals via embryo manipulation and microinjection, particularly animals such as mice, have become conventional in the art. Similar methods are used for production of other transgenic animals. A transgenic founder animal can be identified based upon the presence of a transgene of the invention in its genome and/or expression of mRNA corresponding to a gene of the invention in tissues or cells of the animals. A transgenic founder animal can then be used to breed additional animals carrying the transgene. Moreover, transgenic animals carrying a transgene encoding MDK can further be bred to other transgenic animals carrying other transgenes.

To create a homologous recombinant animal (knockout animal), a vector is prepared which contains at least a portion of a gene of the invention into which a deletion, addition or substitution has been introduced to thereby alter, e.g., functionally disrupt, the gene. The gene can be a human gene, but more preferably, is a non-human homolog of a human gene of the invention (e.g., a homolog of the MDK gene). For example, a mouse gene can be used to construct a homologous recombination polynucleotide, e.g., a vector, suitable for altering an endogenous gene of the invention in the mouse genome. In a preferred embodiment, the homologous recombination polynucleotide is designed such that, upon homologous recombination, the endogenous gene of the invention is functionally disrupted (i.e., no longer encodes a functional protein; also referred to as a “knockout” vector). Alternatively, the homologous recombination polynucleotide can be designed such that, upon homologous recombination, the endogenous gene is mutated or otherwise altered but still encodes functional protein (e.g., the upstream regulatory region can be altered to thereby alter the expression of the endogenous MDK gene). In the homologous recombination polynucleotide, the altered portion of the gene of the invention is flanked at its 5′ and 3′ ends by additional polynucleotide sequence of the gene of the invention to allow for homologous recombination to occur between the exogenous gene carried by the homologous recombination polynucleotide and an endogenous gene in a cell, e.g., an embryonic stem cell. The additional flanking polynucleotide sequence is of sufficient length for successful homologous recombination with the endogenous gene.

Typically, several kilobases of flanking DNA (both at the 5′ and 3′ ends) are included in the homologous recombination polynucleotide. The homologous recombination polynucleotide is introduced into embryonic stem cells by electroporation. The cells in which the introduced gene has homologously recombined with the endogenous gene are selected. The selected cells can then be injected into a blastocyst of an animal (e.g., a mouse) to form aggregation chimeras. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the homologously recombined DNA. Methods for constructing homologous recombination polynucleotides, e.g., vectors, or homologous recombinant animals are well known in the art.

In another embodiment, transgenic non-human animals can be produced which contain selected systems which allow for regulated expression of the transgene. One example of such a system is the cre/loxP recombinase system of bacteriophage PI. Another example of a recombinase system is the FLP recombinase system of Saccharomyces cerevisiae (see e.g., O'Gorman et al., Science 251:1351-1355, 1991). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al., Nature 385:810-813, 1997, and PCT International Publication Nos. WO97/07668 and WO97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

### EXAMPLES

### Example 1

### RNA Isolation and Hybridization to Oligonucleotide Arrays

MRL/MpJ-Fas^{lpr}, MRL/MpJ, NZB×NZW F1, NZB×NZB F1, B6/MRL-Fas^{lpr} C57B16/J, SJL/J, Balb/c, and DBA2/J mice were purchased from Jackson Laboratories (Bar Harbor, Me). Five month old MRL/MpJ-Fas^{lpr} male mice were received as retired breeders. All other mice were obtained at 6 to 8 weeks of age and aged on site. The rapamycin-treated NZB×NZW F1 mice was injected with rapamycin subcutaneously, 5 mg/kg, 3 times per week for 8 weeks, with treatment beginning at 29 weeks of age.

Kidneys from both male and female mice were collected and snap frozen for RNA isolation. One half of each kidney (a longitudinal section of the left kidney and a cross section of the right kidney) was harvested from each mouse in the study. Snap frozen mouse kidney tissue was homogenized using homogenizer suspended in RLT buffer plus 2-mercaptoethanol for 30 to 45 seconds. Total RNA was prepared using the Qiagen Midi Kit following the manufacturer's protocol. RNA was suspended in DEPC-treated water and quantified by OD 280.

Gene expression analysis was performed on individual kidney RNA samples harvested from the following mice: C57BL/6 female mice at 8 weeks (n=3), 20 weeks (n=3) and 32 (n=3) weeks; MRL/MpJ-Fas^{lpr} male at 8 weeks (n=3) and 20 weeks (n=2); MRL/MpJ-Fas^{lpr} female mice at 8 weeks (n=3), 16 weeks and 20 weeks (n=6 combined), MRL/MpJ female mice at 8 (n=3) and 20 weeks (n=3), MRL/MpJ male mice at 8 (n=3) and 24 weeks (n=2), B6/MRL-Fas^{lpr} male at 8 weeks (n=3) and 20 weeks (n=3) and B6/MRL-Fas^{lpr} female mice at 8 weeks (n=3) and 20 weeks (n=3). Thus the total number of individual RNA samples subjected to gene expression analysis using the Affymetrix Gene chip arrays was 46, 21 of which were harvested from lupus nephritis-free stains and the remainder from mice either before, during or after disease onset.

cDNA was synthesized from 5 μg of total RNA from each individual kidney sample using the Superscript Kit (Life Technologies, Rockville, Md.) with modifications described in detail previously Byrne et al. (Byrne, et al., in Current Protocols in Molecular Biology, John Wiley and Sons, Inc, New York, 2000). cDNA was purified using phenol:cloroform:isoamyl alcohol (25:24:1) with a Phage lock gel tube following the Phage lock protocol. Supernatant was collected and cleaned up using ethanol. Sample was resuspended in DEPC-treated water.

In vitro T7 polymerase driven transcription reactions for synthesis and biotin labeling of antisense cRNA, Qiagen Rneasy spin column purification and cRNA fragmentation were carried out in as previously described (Lockhart et al., Nature Biotechnology 14, 1675-80, 1996). GeneChip hybridization mixtures contained 15 μg fragmented cRNA, 0.5 mg/ml acetylated BSA, 0.1 mg/ml herring sperm DNA, in 1×MES buffer in a total volume of 200 μl as per manufactures instructions. Reaction mixtures were hybridized for 16 hr at 45° C. to Affymetrix Mull KsubA and Mull KsubB oligonucleotide arrays. The hybridization mixtures were removed and the arrays were washed and stained with Streptavidin R-phycoerthrin (Molecular Probes, Eugene, Oreg.) using GeneChip Fluidics Station 400 and scanned with a Hewlett Packard GeneArray Scanner following manufactures instructions. Fluorescent data was collected and converted to gene specific difference average using MicroArray Suite software.

### Example 2

### Calculation of Gene Expression Frequency

An eleven member standard curve, comprised of gene fragments derived from cloned bacterial and bacteriophage sequences were spiked into each hybridization mixture at concentrations ranging from 0.5 pM to 150 pM representing RNA frequencies of approximately 3.3 to 1,000 parts per million (ppm). The biotinylated standard curve fragments were synthesized by T7-polymerase driven IVT reactions from plasmid-based templates. The spiked biotinylated RNA fragments serve both as an internal standard to assess chip sensitivity and as standard curve to convert measured fluorescent difference averages from individual genes into RNA frequencies in ppm as described by Hill et al., (Hill et al., Genome Biol. 2. Res 0055.1-0055.13, 2001). Gene expression frequencies from each individual mouse kidney were measured and the expression data subjected to statistical analysis. Array images were processed using the Afmetrix MicroArray Suite 4 software as follows. Raw array image data (.dat files) were reduced to probe feature-level intensity summaries (.cel files). Probe intensities for each message were then summarized using the Affymetrix Average Difference algorithm, and the Affymetrix Absolute Decision metric was computed (Absent, Present, or Marginal) for each gene. The Average Difference values were converted to estimates of absolute message abundance (in parts per million) by the scaled frequency method as previously described by Hill et al. Briefly, Average Difference values were globally scaled to make the 2% trimmed mean average difference equal for all arrays. Standard curves from spiked cRNAs in each hybridization were then pooled from all arrays, and fitted by a linear calibration function passing through the origin. The scaled Average Difference values from all arrays were multiplied by the slope of this fitted calibration function to give initial frequency estimates. Frequencies smaller than the estimated sensitivity of each array were then adjusted to the average of the frequency and the sensitivity, in order to eliminate negative readouts. Due variation in sensitivity among probe sets for different messages, frequencies should be viewed as estimates, and inter-gene comparisons of frequencies should be interpreted cautiously.

### Example 3

### Selection of Genes in Analysis Set

The detection of any gene was deemed unreliable if it was not called present in at least 50% of samples from at least one group and was eliminated from the set of genes under analysis. Similarly, in order to avoid conclusions dependent on the lower (and less reliable by Taqman PCR) limits of the standard curve, any gene with average frequency not greater than 9 ppm in at least one group was eliminated from analysis. These operations resulted in a list of 5,285 tiled oligonucleotides representing the set of genes to be surveyed for MRL strain-dependent gene expression differences.

### Example 4

### Flagging of Potential Age, Gender and fas^{lpr} Dependent Gene Expression Differences

Average fold change (AFC) was obtained by dividing the average frequency of one group by the average of the other group. To identify genes whose expression levels are influenced by gender, the AFC between male and female groups was calculated for each of the six groups of male and female mice listed above. All genes with fold change differences consistent between male and female mice in each group combination were flagged as demonstrating a possible gender-influenced. Genes with AFC>1.5 between 8 and 32 week old C57BL/6 (disease free) were flagged as “possibly age-influenced”. Gene with AFC>1.5 between C57BL/6 and C57BL/6-Fas^{lpr} were flagged as demonstrating an effect of the Fas^{lpr} mutation that did not depend on the disease prone MRL genetic background. Genes identified through these processes as demonstrating possible gender, age and Fas^{lpr} influences on expression frequency were flagged but retained on the list of genes surveyed for influences related to the MRL genetic background.

### Example 5

### Quantitative Reverse Transcriptase-polymerase Chain Reaction (Taqman Analysis)

Quantitative RT-PCR was performed using RNA samples from murine kidneys were treated with 10U of RQ1 DNase I (Promega, Madison, Wis., USA) for 30 minutes at 37° C. 10 ng of total RNA was reverse transcribed and amplified in a single tube assay using the TaqMan® One Step PCR Master Mix Reagent Kit (Applied BioSystems, Foster City, Calif.) with gene specific sense and anti-sense primers and a probe fluorecently labeled at the 5′ end with 6-carboxy-fluorscein (6-FAM). Amplification was performed using the ABI Prism 7700 sequence detection system as described by the manufacturer. Primers and fluorescently labeled probes were generated using Primer Express software (Applied BioSystems, Foster City, Calif.). Sequence-specific amplification was detected as an increased fluorescent signal of 6-FAM during the amplification cycle. Quantitation of gene-specific message levels was based on a comparison of the fluorescent intensity in the unknown mRNA sample to the fluorescent intensity from a standard curve of known mRNA levels. Amplification of the gene for cyclophilin was performed on all samples tested to control for variations in RNA amounts. All genes were subsequently normalized to cyclophilin mRNA levels. Levels of gene-specific messages were graphed as normalized message units as determined from the standard curve. A no template control was included in each amplification reaction to control for contaminating templates. The primers and probe used in the Taqman analysis are listed below:

| | |
|---|---|
| Forward primer: | |
| 5′-CGGTGGGCAAGCGAAGT-3′; | (SEQ ID NO:53) |
| Reverse Primer: | |
| 5′-CCCCTGGTCTAGGCCTGTCT-3′; | (SEQ ID NO:54) |
| Probe: | |
| 5′-AGAGCTGACAGGCTGCGAGAGGGA-3′. | (SEQ ID NO:55) |

1. A method comprising the steps of:
detecting an expression level of midkine gene in a biological sample isolated from a mammal of interest; and
comparing the expression level to a reference expression level of said midkine gene in at least one control sample.

2. The method of claim 1, wherein said at least one contro sample is isolated from at least one contro mammal, wherein said at least one control mammal does not have systemic lupus erythematosus or lupus nephritis.

3. The method of claim 2, wherein the mammal of interest has systemic lupus erythematosus or lupus nephritis.

4. The method of claim 2, wherein the expression level and the reference expression level are detected using an antibody directed against a product of said midkine gene.

5. The method of claim 2, wherein the expression level and the reference expression level are detected by measuring the level of an RNA transcript of said midkine gene.

6. The method of claim 2, wherein the biological sample is selected from the group consisting of a tissue sample, a urine sample, and a blood sample.

7. The method of claim 2, wherein the biological sample and said at least one control sample are kidney samples.

8. The method of claim 2, wherein the mammal of interest is a human.

9. A pharmaceutical composition for preventing or treating systemic lupus erythematosus or lupus nephritis, comprising a pharmaceutically acceptable carrier and an agent that modulates a midkine activity or midkine gene expression.

10. The pharmaceutical composition of claim 9, wherein the agent inhibits said midkine activity or midkine gene expression.

11. The method of claim 9, wherein the agent is selected from the group consisting of a polypeptide, a polynucleotide, a polysaccharide, a small organic molecule, and an inorganic molecule.

12. The pharmaceutical composition of claim 11, wherein the agent is an antibody that binds specifically to a midkine gene product.

13. The pharmaceutical composition of claim 11, wherein the agent is an antisense polynucleotide to midkine gene.

14. The pharmaceutical composition of claim 9, wherein the agent is a gene therapy vector capable of producing in vivo a polypeptide or a polynucleotide that modulates said midkine activity or midkine gene expression.

15. The pharmaceutical composition of claim 9, wherein the agent is a ploynucelotide capable of inhibiting said midkine gene expression by RNAi.

16. The pharmaceutical composition of claim 15, wherein the polynucleotide comprises a siRNA sense strand or a siRNA antisense strand selected from Table 2.

17. A method comprising the step of introducing into a mammal in need thereof an effective amount of the pharmaceutical composition of claim 10.

18. A method of identifying an agent capable of binding to midkine or a variant thereof, comprising:
contacting a polypeptide comprising an amino acid sequence recited in SEQ ID NO:1 or a variant of the polypeptide with a candidate agent; and
determining a binding affinity of the candidate agent to said polypeptide or the variant of said polypeptide.

19. The method of claim 18, wherein said polypeptide, the variant of said polypeptide, or the candidate agent includes a label group.

20. A method of identifying an agent capable of modulating an activity of midkine or a variant thereof, comprising:
contacting a polypeptide comprising the amino acid sequence of SEQ ID NO:1 or a variant of said polypeptide with a candidate agent; and
comparing an activity of said polypeptide or the variant of said polypeptide in the presence of said candidate agent to an activity of said polypeptide or the variant of said polypeptide in the absence of said candidate agent.

21. A kit for diagnosing systemic lupus erythematosus or lupus nephritis, said kit comprising at least one of:
(a) a polynucleotide probe capable of hybridizing under stringent conditions to a polynucleotide encoding the amino acid sequence depicted in SEQ ID NO:1, or the complement thereof, and
(b) an antibody capable of specifically binding to the amino acid sequence depicted in SEQ ID NO:1.
